(19) United States
(12) Patent Application Publication
(10) Pub. No.: US 2013/0345790 A1
(43) Pub. Date: Dec. 26, 2013

(54) MEDICAL DEVICE FOR IMPLANTATION INTO LUMINAL STRUCTURES

(71) Applicant: Robert J. Cottone, Davie, FL (US)

(72) Inventor: Robert J. Cottone, Davie, FL (US)

(73) Assignee: OrbusNeich Medical Inc., Fort Lauderdale, FL (US)

(21) Appl. No.: 13/980,951

(22) PCT Filed: Jan. 27, 2012
(86) PCT No.: PCT/US12/23041

§ 371 Date: Sep. 11, 2013

Related U.S. Application Data
(60) Provisional application No. 61/436,793, filed on Jan. 27, 2011.

(51) Int. Cl. A61F 2/89

(52) U.S. Cl. CPC A61F 2/89
USPC 623/1.16

(57) ABSTRACT

The present invention relates to expandable stents with a geometric design that exhibits a high degree of flexibility and significant radial strength. The stents of the present invention comprise a generally cylindrically shaped main body having a plurality of expandable first and second circumferential elements. When the stent is expanded, the second circumferential element forms a ring- or hoop-like structure. In certain embodiments, the main body contains a plurality of expandable first, second and third circumferential elements where both the second and third circumferential elements form a ring- or hoop-like structure when expanded. The stent may further comprise an end zone that caps the main body, or two end zones positioned at both ends of the main body.

### CROSS-REFERENCE TO RELATED APPLICATIONS

This application claims priority to U.S. Provisional Application No. 61/436,793 filed on Jan. 27, 2011.

### FIELD OF THE INVENTION

The present invention relates to stents. In particular, the present invention relates to geometric designs of stents which exhibit a high degree of radial strength and flexibility.

### BACKGROUND OF THE INVENTION

Stents are scaffolds which are positioned in diseased vessel segments to support the vessel walls. During angioplasty, stents are used to repair and reconstruct blood vessels. Placement of a stent in the affected arterial segment prevents elastic recoil and closing of the artery. Stents also prevent local dissection of the artery along the medial layer. Physiologically, stents may be placed inside the lumen of any space, such as an artery, vein, bile duct, urinary tract, alimentary tract, tracheobronchial tree, cerebral aqueduct or genitourinary system. Stents may also be placed inside the lumen of non-human animals, such as primates, horses, cows, pigs and sheep.

In general, there are two types of stents: self-expanding and balloon-expandable. Self-expanding stents automatically expand once they are released and assume a deployed, expanded state. A self-expanding stent is placed in the vessel by inserting the stent in a compressed state into the affected region, e.g., an area of stenosis. Compression or crimping of the stent can be achieved using crimping equipment (see, http://www.machinesolutions.org/stent_crimping.htm, April, 2009). The stent may also be compressed using a tube that has a smaller outside diameter than the inner diameter of the affected vessel region. Once the compressive force is removed or the temperature raised, the stent expands to fill the lumen of the vessel. When the stent is released from confinement in the tube, the stent expands to resume its original shape, in the process becoming securely fixed inside the vessel against the wall.

A balloon-expandable stem is expanded using an inflatable balloon catheter. Balloon-expandable stents may be implanted by mounting the stent in an unexpanded or crimped state on a balloon segment of a catheter. The catheter, after having the crimped stent placed thereon, is inserted through a puncture in a vessel wall and moved through the vessel until it is positioned in the portion of the vessel that is in need of repair. The stent is then expanded by inflating the balloon catheter against the inside wall of the vessel. Specifically, the stent is plastically deformed by inflating the balloon so that the diameter of the stent is increased and the stent expanded.

There are functional limitations common to many stents. These include, for example, comparative rigidity of the stent in a crimped as well as deployed state, and limited flexibility making delivery and placement in narrow vessels difficult. The present invention provides a geometric design for a stent that offers both a high degree of flexibility and significant radial strength. The design of this stent also allows it to be inserted into small diameter vessels having tortuous vascular anatomy.

### SUMMARY OF THE INVENTION

The present invention provides for a stent comprising at least one bioabsorbable polymer, and comprising a plurality of first circumferential elements and a plurality of second circumferential elements. The first circumferential elements and the second circumferential elements are arranged in an alternating pattern. Adjacent first and second circumferential elements are connected by at least one connection element. The circumference of the first circumferential element is greater than the circumference of the second circumferential elements. The first circumferential element comprises a plurality of undulations. The second circumferential element comprises a hoop-like structure when expanded. The stent may be crimped or expanded.

The present invention further provides for a stent comprising at least one bioabsorbable polymer, and comprising: a plurality of first circumferential elements, a plurality of second circumferential elements and a plurality of third circumferential elements. The first, second and third circumferential elements form a group which is repeated at least three times. Adjacent first and second circumferential elements are connected by at least one connection element; adjacent first and third circumferential elements are connected by at least one connection element; adjacent second and third circumferential elements are connected by at least one connection element. The circumference of the first circumferential element is greater than the circumference of the second and third circumferential elements. The first circumferential element comprises a plurality of undulations. The second circumferential element comprises a hoop-like structure when expanded; the third circumferential element comprises a hoop-like structure when expanded. The stent may be crimped or expanded.

At least one undulation in the first circumferential element comprises at least two segments forming at least one angle which may range from about 30° to about 180° or from about 60° to about 150° when the stent is unexpanded. The segments may be equal or unequal in length. The segment may be linear or curvilinear.

The first circumferential element may comprise a plurality of first undulations and a plurality of second undulations. The first circumferential element may comprise a plurality of first undulations, a plurality of second undulations and a plurality of third undulations.

Adjacent first and second circumferential elements may be connected by two or three connection elements. Adjacent first circumferential element and second circumferential element may be connected by at least one first strut, or may be directly connected. The second circumferential element may be in a sinusoidal pattern when crimped. The second circumferential element may further comprise at least one indentation, such as a notched structure.

The stent may further comprise at least one end zone positioned at either end or both ends of the stent where the end zone comprises a plurality of cylindrical elements. The cylindrical element may be in a sinusoidal pattern when unexpanded. The end zone may be attached to the first circumferential element by at least one second strut. The end zone may be directly attached to the first circumferential element. The end zone may further comprise at least one radiopaque marker.

Adjacent first and second circumferential elements may be connected by two connection elements. Adjacent first and third circumferential elements may be connected by two connection elements. Adjacent second and third circumferential elements may be connected by six connection elements. Adjacent second circumferential element and third circumferential element may be connected by at least one first strut. Adjacent first circumferential element and second circumferential element may be directly connected. Adjacent first circumferential element and third circumferential element may be directly connected.

The second circumferential element may be in a sinusoidal pattern when unexpanded. The third circumferential element may be in a sinusoidal pattern when unexpanded.

### BRIEF DESCRIPTION OF THE DRAWINGS

FIG. 1 is a planar view showing a portion of an embodiment of the stent design in an unexpanded form.

FIG. 2 is a planar view showing an alternative embodiment of the stent in an unexpanded form, highlighting the connection elements (or bridging elements), or lack thereof, as well as different ends of the stem.

FIG. 3 is a planar view showing an alternative embodiment of the stent in an unexpanded form.

FIG. 4 is a planar view showing an alternative embodiment of the stent in an unexpanded form, which incorporates radiopaque markers.

FIG. 5 shows a planar view of two embodiments of the stent in an unexpanded form, highlighting the undulations of the first circumferential elements (boxed regions).

FIG. 6 shows a planar view of another embodiment of the stent in an unexpanded form.

FIG. 7 is a planar view showing an alternative embodiment of the stent in an unexpanded form, which incorporates radiopaque markers. FIG. 7 also shows the double end-ring on the right with horizontal connection struts and marker dot place holders, as well as the double end-ring on the left with slanted offset connection struts.

FIG. 8 is a planar view showing an alternative embodiment of the stent in an unexpanded form, which incorporates radiopaque markers.

FIG. 9 is a planar view showing an alternative embodiment of the stent in an unexpanded form. The end of stent on the left comprises a double ring structure; the end of the stent on the right comprises a single end ring structure.

FIG. 10 is a planar view showing an alternative embodiment of the stent in an unexpanded form, which incorporates radiopaque markers.

FIG. 11 is a planar view showing an alternative embodiment of the stent in an unexpanded form, which incorporates radiopaque markers.

FIG. 12 is a planar view showing an alternative embodiment of the stent in an unexpanded form, which incorporates radiopaque markers.

FIG. 13A is a planar view showing an alternative embodiment of the stem in an unexpanded form, which incorporates radiopaque markers. FIGS. 13B and 13C show the enlarged undulations.

FIG. 14A is a planar view showing an alternative embodiment of the stem in an unexpanded form, which incorporates radiopaque markers. FIGS. 14B and 14C show the enlarged undulations.

FIG. 15 is a planar view showing an alternative embodiment of the stent in an unexpanded form, which incorporates radiopaque markers.

FIG. 16 is a planar view showing an alternative embodiment of the stent in an unexpanded form, which incorporates radiopaque markers.

FIG. 17A is a planar view showing an alternative embodiment of the stent in an unexpanded form, which incorporates radiopaque markers. FIGS. 17B and 17C show the enlarged undulations.

FIG. 18 is a planar view showing an alternative embodiment of the stent in an unexpanded form, which incorporates radiopaque markers.

FIG. 19 is a planar view showing an alternative embodiment of the stent in an unexpanded form, which incorporates radiopaque markers.

FIG. 20 is a planar view showing an alternative embodiment of the stent in an unexpanded form, highlighting the undulations and connection elements.

FIG. 21 is a planar view showing an alternative embodiment of the stent in an unexpanded form, which incorporates radiopaque markers.

FIG. 22 is a planar view showing an alternative embodiment of the stent in an unexpanded form, which incorporates radiopaque markers.

FIG. 23A is a planar view showing an alternative embodiment of the stent in an unexpanded form, which incorporates radiopaque markers. FIGS. 23B and 23C show the enlarged undulations.

FIG. 24 is a planar view showing an alternative embodiment of the stem in an unexpanded form, highlighting the notched single ring structure.

FIG. 25 is a planar view showing an embodiment of the stent in an unexpanded form.

FIG. 26 is a perspective view showing an alternate embodiment of the stent in an unexpanded form.

FIG. 27 is a planar view showing an embodiment of the stent in an unexpanded form, highlighting the first and second circumferential elements.

FIG. 28 is a planar view showing an alternative embodiment of the stem in an unexpanded form.

FIG. 29 shows a planar view of another embodiment of the stent in an unexpanded form.

FIG. 30 is a perspective view showing an alternative embodiment of the stent in an expanded form.

FIG. 31 is a planar view showing an alternative embodiment of the stent in an expanded form.

FIGS. 32A-F show various embodiments of the connection elements.

FIG. 33A depicts a partial top view of expanded hoop or ring and circumferential (FIG. 33B) bioabsorbable elements of a stent embodiment. FIG. 33C illustrates a hoop or ring clement of a bioabsorbable stent showing how radial/transverse load is distributed through a ring structure. As illustrated such structure provides a better distribution of forces keeping such stent open under forces that might otherwise cause deformation of the stent. FIG. 33D illustrates a hoop undergoing progressive radial expansion. FIG. 33E shows the stent ring undergoing increasing radial expansion. FIG. 33F illustrates the phenomena referred to as “necking” as the cross section of the ring decreases in a specific section of the circumferential element and crystallization spreads laterally around the ring.

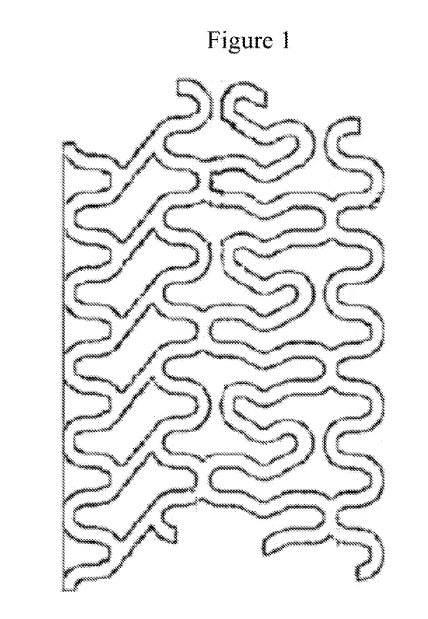

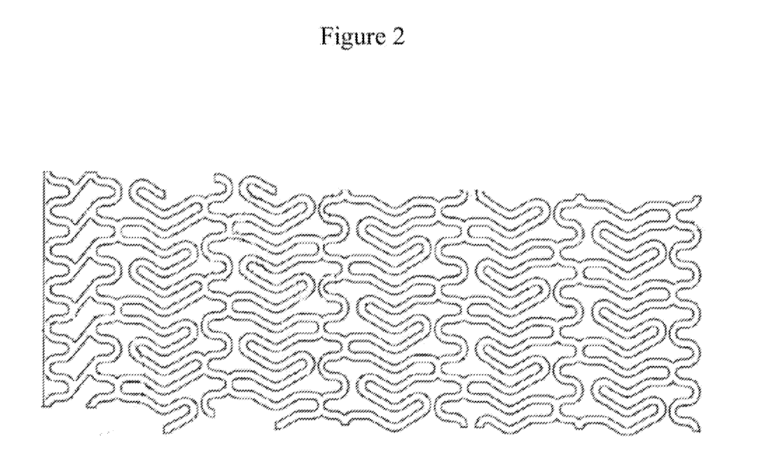

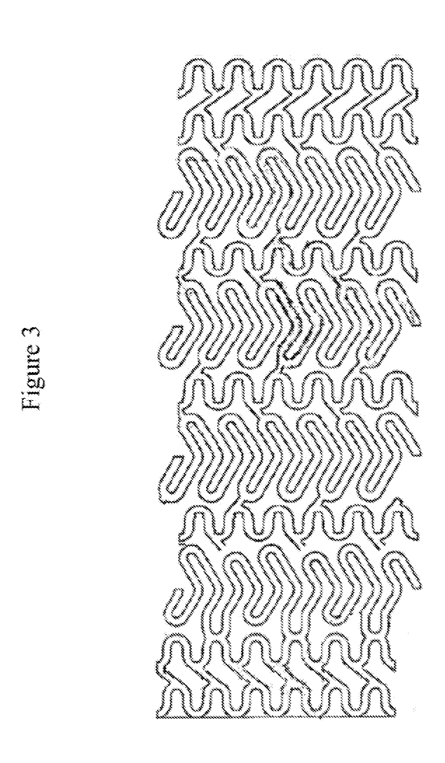

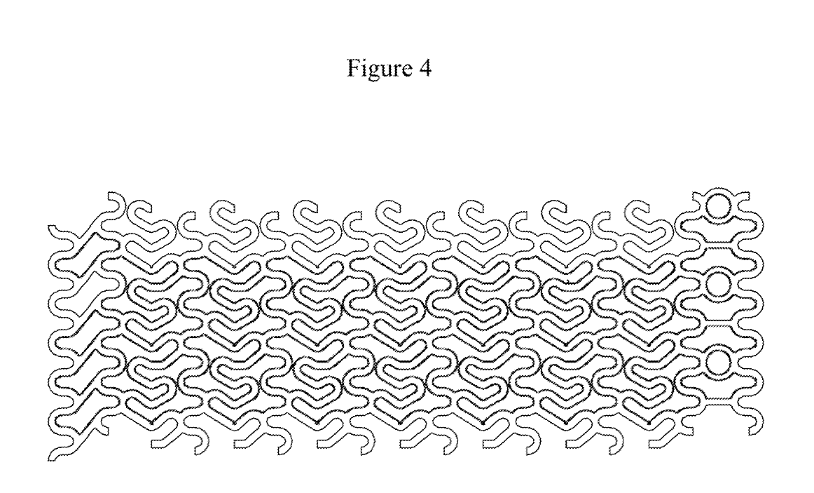

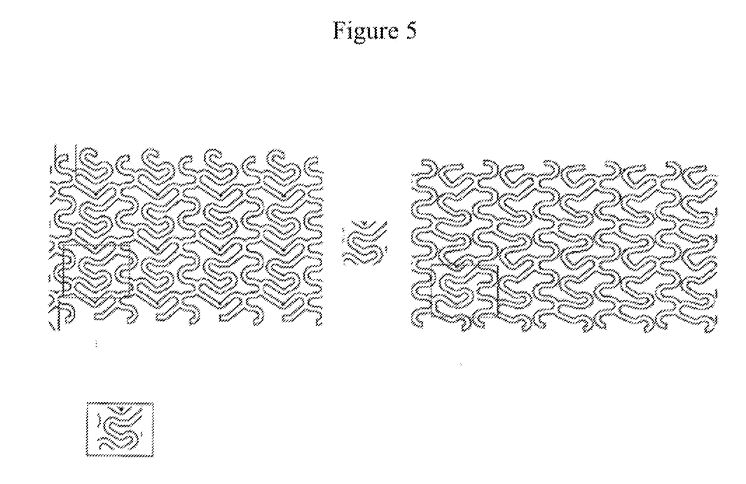

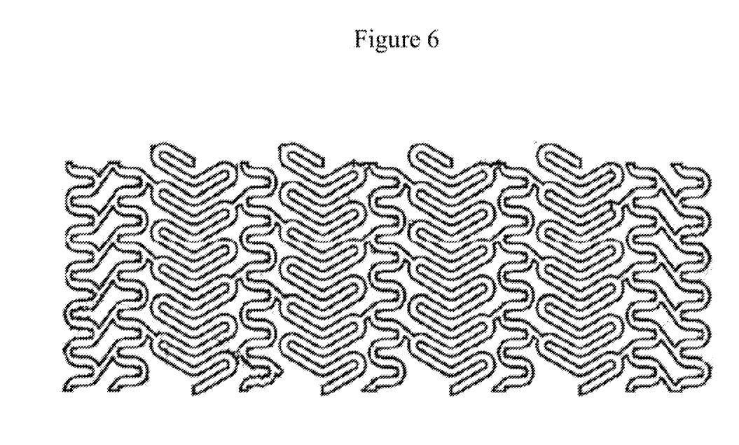

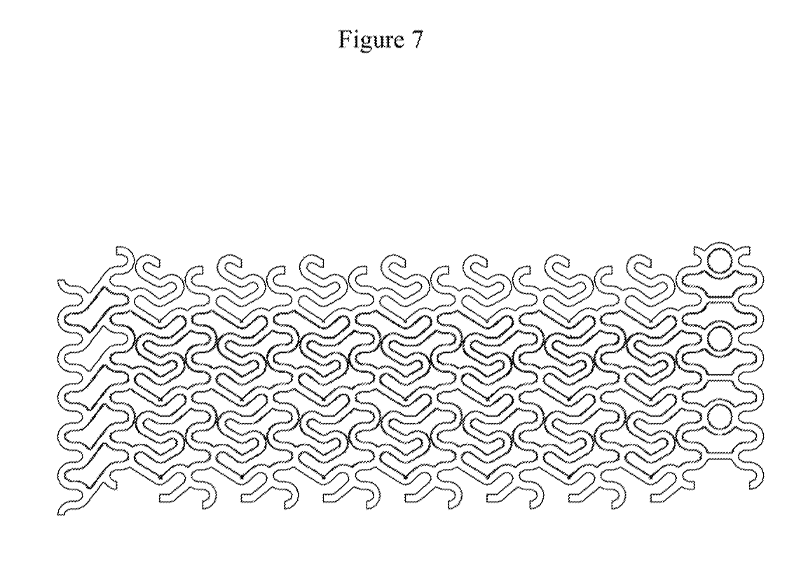

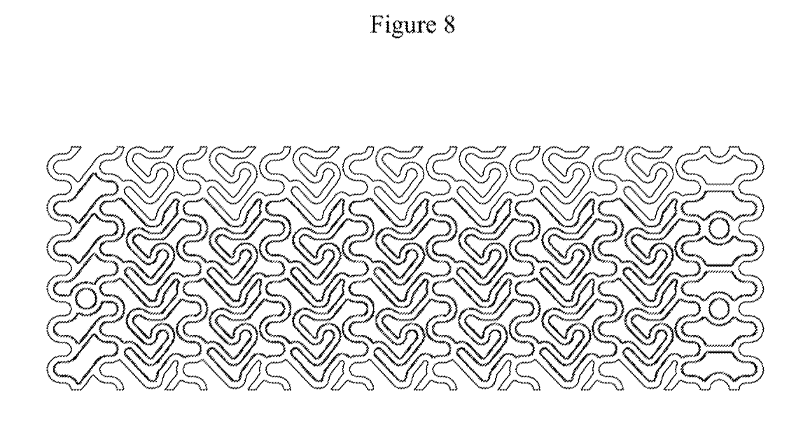

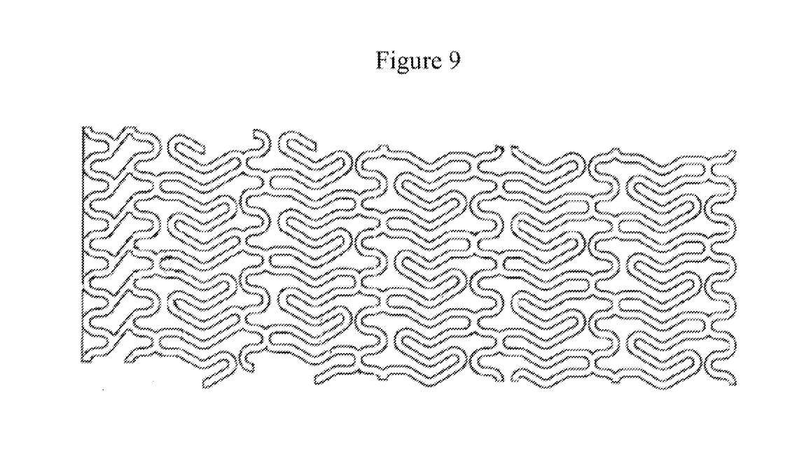

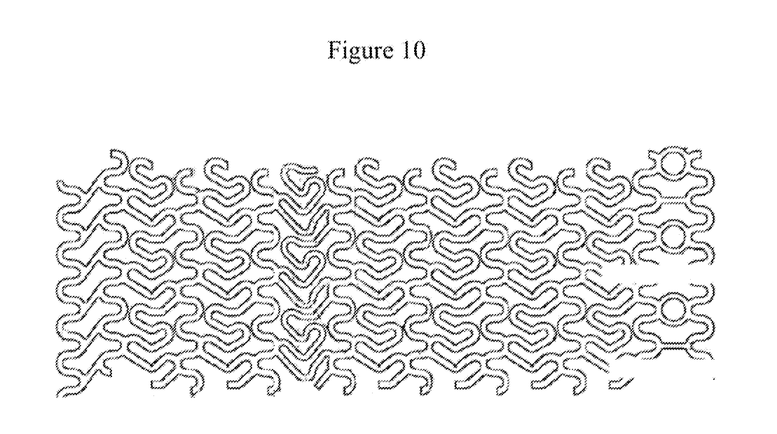

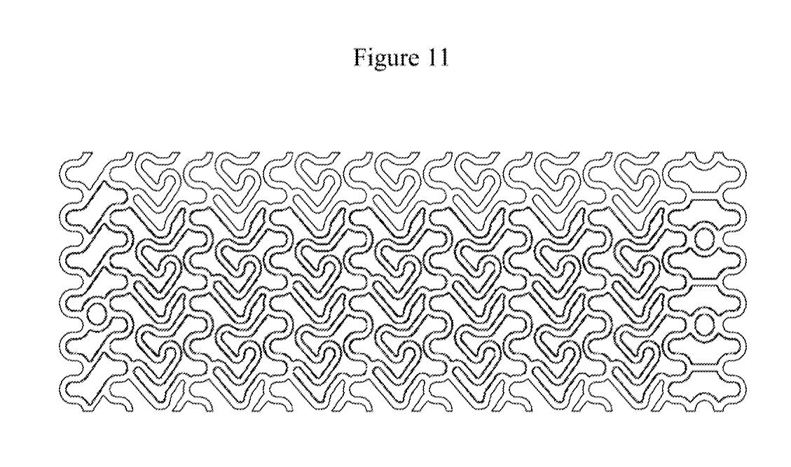

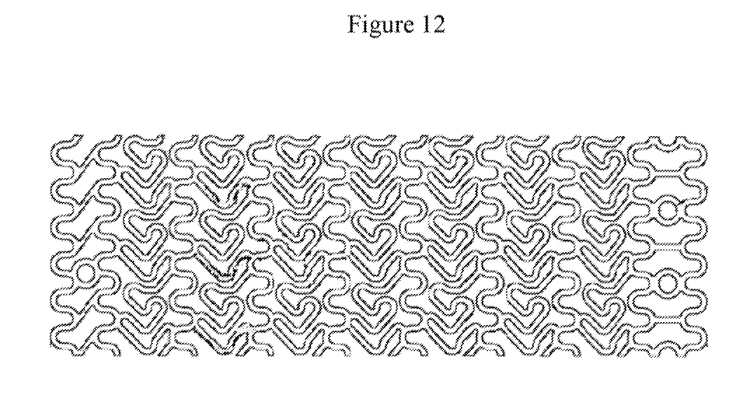

### DETAILED DESCRIPTION OF THE INVENTION

The present invention relates to expandable stents with a geometric design that exhibits a high degree of flexibility and significant radial strength. The stents of the present invention comprise a generally cylindrically shaped main body having a plurality of expandable first and second circumferential elements. When the stent is expanded, the second circumferential element forms a ring- or hoop-like structure. Therefore, when expanded, the stent comprises a stack of single rings separated by expanded first circumferential elements. In certain embodiments, the main body contains a plurality of expandable first, second and third circumferential elements where both the second and third circumferential elements form a ring- or hoop-like structure when expanded. Therefore, when expanded, the stent comprises a stack of double rings separated by expanded first circumferential elements. In certain embodiments, the stent may comprise a plurality of triple rings separated by expanded first circumferential elements. The stent may further comprise an end zone that caps the main body, or two end zones positioned at both ends of the main body.

In certain embodiments, the main body of the stent may contain a plurality of first and second circumferential elements which are arranged in an alternating pattern. The circumferential elements have cylindrical axes that are collinear with the cylindrical axis of the main body. The circumferential elements may be substantially wave-like in form providing a series of alternating valleys and peaks. The circumferential elements may also take other forms, such as zigzag patterns. When a radial expanding force is applied to the stent, the circumferential elements expand radially and elongate circumferentially. Conversely, when an external compressive force is exerted on the stent, the circumferential elements contract radially and shorten circumferentially. The circumference of the first circumferential element can be greater than the circumference of the second circumferential elements. Alternatively, the circumference of the first circumferential element can be less than or equal to the circumference of the second circumferential elements. The first and second circumferential elements may be different or substantially identical.

A first circumferential element may comprise a plurality of meandering elements or undulations. An undulation may take the shape of a stylized S, Z, L (I), M, N, W, etc. An undulation may also take any other suitable configurations. The undulations may be joined together to form a pattern. When the stent is crimped, the pattern may be repeating or non-repeating. The undulations within a circumferential element may be identical or different. For example, a circumferential element may comprise a plurality of first undulations and a plurality of second undulations. A circumferential element may also comprise a plurality of first undulations, a plurality of second undulations and a plurality of third undulations. More types of undulations within a circumferential element are further encompassed by the invention. The undulations may be joined together to form an alternating pattern or other repeating patterns. Non-limiting examples of the repeating patterns include, a sinusoidal wave form, a square wave form, a rectangular wave form, a triangle wave form, a spiked wave form, a trapezoidal wave form and a saw-tooth wave form. The undulations in a circumferential element may also be joined together to produce a non-repeating pattern. Patterns that can be used in the present invention include any suitable pattern that enables the circumferential clement to expand when a radial expanding force is exerted on the stent or collapse when an external compressive force is applied.

An undulation may have at least one amplitude. As used herein, the amplitude of an undulation is defined by an axial distance between the valley (or one of the valleys) and peak (or one of the peaks) of the undulation. When a radial expanding force is applied to the stem, the undulation contracts in amplitude. Conversely, when an external compressive force is exerted on the stent, the undulation increases in amplitude. When a circumferential element contains more than one undulations, the amplitude of the undulations may be identical or different. In a second circumferential element, each peak may be axially spaced a similar distance from each valley such that the undulations in the second circumferential element have identical amplitude. Alternatively, the amplitude of the undulations of the second circumferential element may vary. The amplitude of the undulations of the first circumferential element may be greater than, equal to or less than the amplitude of the undulations of the second circumferential element.

A first circumferential element may contain identical or different undulations. For example, when the undulations are not identical, there may be a plurality of first undulations and a plurality of second undulations arranged in an alternating pattern in a first circumferential element. There may also be a plurality of first undulations, a plurality of second undulations and a plurality of third undulations in a first circumferential element. The number of types of undulations in a first circumferential element may range from 1 to 20, from I to 15, from 2 to 10, or from 2 to 6.

A second circumferential element may contain a plurality of undulations forming a repeating or non-repeating pattern. For example, when crimped, the second circumferential element may be in a sinusoidal pattern. As described above, the second circumferential element may take any suitable configuration. In one embodiment, the second circumferential element may comprise a ring- or hoop-like structure when expanded where the ring or hoop is substantially in the same plane. As used herein, the term “plane” is defined as a theoretical two-dimensional unit that is cutting substantially orthogonal to the cylindrical axis of the stent. A second circumferential element may contain identical or different undulations. For example, when the undulations are not identical, there may be a plurality of first undulations and a plurality of second undulations.

An undulation may comprise one segment or at least two segments with at least two adjacent segments forming an angle. The segments may be linear or curvilinear. When a segment is curvilinear, the degree of curvature may vary. A segment may be concave or convex. A segment may contain solely linear portions joined together, or solely curved portions joined together. Alternatively, a segment may contain both linear portions and curved portions that are joined together. The linear portion may be parallel to the cylindrical axis of the stent, or lie at an angle ranging from about 0° to about 60°, or from about 0° to about 45° with respect to the cylindrical axis of the stent. The segment may comprise at least one bend placed at selected points along its length. For example, a segment may take the shape of a stylized n, C, U, V, etc. A segment may also be in the shape of a loop where the loop may be circular or semicircular. The segment can essentially assume any suitable configuration.

At least two segments can be joined together to form an undulation or meandering element. An undulation may contain N segments (N is any positive integer: 1, 2, 3, 4, 5, etc.) with adjacent 0 to N-1 pair(s) of segments forming from 0 to N-1 angles. For example, in a first circumferential element, an undulation may comprise two opposing angled segments forming an angle. An undulation can contain three segments forming 0, 1 or 2 angles. An undulation can contain four segments forming 0, 1, 2 or 3 angles. Higher numbers of undulations and angles formed by adjacent segments are also encompassed by the invention. Within an undulation, the angles may be constant or may vary. In one embodiment, in the first circumferential element, there are two opposing angled segments in each undulation where the segments form an angle in each undulation. Within a first circumferential element, the angle(s) formed by the segments may be constant or may vary among the undulations. The length, width and thickness of the segments within an undulation in the first circumferential element may be equal or unequal. In certain embodiments, a circumferential element comprises solely segments.

Additionally, adjacent undulations may form an angle. In a first circumferential element, the angles formed by adjacent undulations may be constant or may vary. The angle formed by adjacent segments in an undulation may be greater than, equal to or less than the angle formed by adjacent undulations. When the stent is crimped, the angle may range from about 30° to about 180°, from about 45° to about 160°, from about 60° to about 150°, or from about 90° to about 120°.

In certain embodiments, at least two adjacent segments form a first angle; at least two adjacent undulations form a second angle.

The filament width of the circumferential elements may range from about 0.05 mm to about 2.5 mm, from about 0.05 mm to about 1.3 mm, from about 1 mm to about 2 mm, from about 1.5 mm to about 2.5 mm, from about 0.05 mm to about 1.5 mm, from about 0.05 mm to about 1 mm, from about 0.05 mm to about 0.5 mm, from about 0.05 mm to about 0.3 mm, from about 0.08 mm to about 0.25 mm, from about 0.1 mm to about 0.25 mm, from about 0.12 mm to about 0.2 mm, about 0.18 mm, about 0.20 mm, or about 0.13 mm.

Adjacent circumferential elements may be connected by at least one connection element. For example, a first circumferential element can be connected to an adjacent second circumferential element by at least one connection element. Adjacent circumferential elements may be connected by 1 connection element, 2, 3, 4, 5, 6, 7, 8, 9, 10 connection elements. Higher numbers of connection elements are also encompassed by the present invention.

A connection element may take various configurations. A connection element can simply be an adjoining point/region of adjacent circumferential elements. In this case, adjacent circumferential elements (e.g., a first circumferential element and an adjacent second circumferential element, a first circumferential element and an adjacent third circumferential element, or a second circumferential element and an adjacent third circumferential element) are directly connected. A connection element can comprise a strut. For example, adjacent circumferential elements (e.g., a first circumferential element and an adjacent second circumferential element, a first circumferential element and an adjacent third circumferential element, or a second circumferential element and an adjacent third circumferential element) may be connected by at least one first strut. In certain embodiments, a connection element comprises a first strut diagonally interconnecting opposite sides of undulations in adjacent circumferential elements. A connection element can comprise two or more struts. For example, adjacent circumferential elements may also be connected by at least one first strut and at least one second strut. In one embodiment, a connection element comprises two intersecting struts. The struts may assume a variety of angles relative to the cylindrical axis of the stent, including, 0-20°, 20-40° and 40-60° (the angle of these struts may be positive or negative relative to the cylindrical axis of the stent). The struts may be straight or curvilinear. The curvilinear strut may be concave and convex with curvature present at selected portions of the strut. The degree of curvature may vary.

A connection element may connect the peak of an undulation in a circumferential element to the valley of an undulation in the adjacent circumferential element. A connection element may connect the peak (or valley) of an undulation in a circumferential element to the peak (or valley) of an undulation in the adjacent circumferential element. Essentially any regions of adjacent circumferential elements can be connected by a connection element.

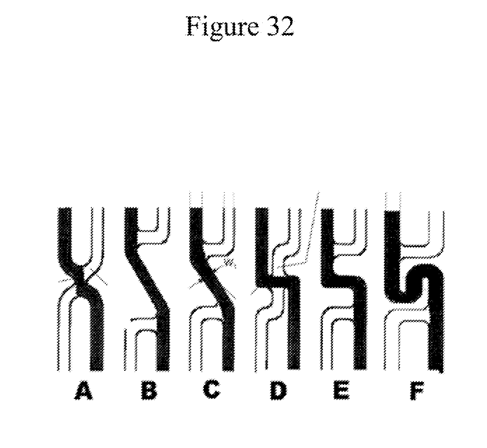

The form, number and location of the connection elements may be adapted to result in desired stent properties. The connection elements may be H-shaped, S-shaped, O-shaped, I-shaped, L-shaped, M-shaped, X-shaped, Y-shaped, etc. Other types of connection elements are also feasible. Examples of other embodiments of the connection elements which could be used in the present invention are shown in FIG. 32.

The number of connection elements between adjacent circumferential elements may be modified to suit the flexibility of the stent. For example, the fewer connection elements between adjacent circumferential elements, the more flexible the stent may be. When there are more than one connection elements between adjacent circumferential elements, the connection elements may be positioned symmetrically or asymmetrically at radial positions along the circumference of the stent. If the connection elements are positioned symmetrically, the radial distance between each pair of connection elements, e.g., A-B and B-C, is equal. The radial positions listed for the connection elements here are only provided for illustration purposes and the connection elements may be positioned by one of ordinary skill in the art without undue experimentation at any point along the circumference of the stent. For example, the positioning of the connection elements may be determined by dividing 360° by n where n is the number of connection elements between adjacent circumferential elements. Where n=3, the connection elements may be positioned symmetrically at approximately 120° intervals around the circumference of the stent. When there are two equally spaced connection elements between adjacent circumferential elements, they are situated approximately 180° with respect to one another. In other words, the two connection elements are oppositely oriented with respect to one another.

When adjacent circumferential elements are connected by at least one first strut, the number of the first struts may vary. For example, there may be 1, 2, 3, 4, 5 or 6 first struts connecting adjacent circumferential elements. Higher numbers of first struts are also encompassed by the present invention. The first struts may assume a wide variety of angles relative to the cylindrical axis of the stent, including, 0°-20°, 20°-40°, 40°-60°, 0-70°, 20°-60°, 30°-55° and 45°-50°. The angles may also be negative, i.e., fall on the opposite side of the cylindrical axis of the stent. The first struts may have the same or different angles with respect to one another.

In another embodiment, the main body of the present stent comprises a plurality of first circumferential elements, a plurality of second circumferential elements and a plurality of third circumferential elements. The first, second and third circumferential elements are arranged in a repeating pattern. The first, second and third circumferential elements form a group which is repeated at least two times, at least three times, at least four times, at least five times, two times, three times, four times, five times, six times. In other words, in the group, a first circumferential element is adjacent to a second circumferential element, and a second circumferential is adjacent to a third circumferential element. The main body may contain two repeats, three repeats, four repeats, five repeats or six repeats, of the group which has first, second and third circumferential elements. The circumference of the first circumferential element is greater than the circumference of the second and third circumferential elements. Alternatively, the circumference of the first circumferential element may be equal to or less than the circumference of the second and third circumferential elements. A first circumferential element may comprise a plurality of undulations or meandering elements. An undulation may comprise one segment or at least two segments with at least two adjacent segments forming an angle. The segments may be linear or curvilinear. At least two segments can be joined together to form an undulation or meandering element. See above for detailed descriptions on the segments and undulations, as well as the angles formed by segments or undulations.

The circumferential element comprises a plurality of undulations. In the first circumferential element, each undulation has at least two segments forming an angle. In the second circumferential elements, each peak may be axially spaced a similar distance from each valley such that each second circumferential element has a constant amplitude. Alternatively, the amplitude of the second circumferential element may vary. In the third circumferential elements, each peak may be axially spaced a similar distance from each valley such that each third circumferential element has a constant amplitude. Alternatively, the amplitude of the third circumferential element may vary. The second and third circumferential elements may be different or substantially identical.

The second circumferential element may contain a plurality of undulations forming a repeating or non-repeating pattern. For example, when crimped, the second circumferential element may be in a sinusoidal pattern. The second circumferential element may take any suitable configuration. In one embodiment, the second circumferential element may comprise a hoop-like structure when expanded where the hoop is substantially in the same plane.

The third circumferential element may contain a plurality of undulations forming a repeating or non-repeating pattern. For example, when crimped, the third circumferential element may be in a sinusoidal pattern. The third circumferential element may take any suitable configuration. In one embodiment, the third circumferential element may comprise a hoop-like structure when expanded where the hoop is substantially in the same plane.

A first circumferential element is connected to an adjacent second or third circumferential element by at least one connection element. Similarly, a second circumferential element is connected to an adjacent third circumferential element by at least one connection element. For example, the second and third circumferential elements may be connected by at least one first strut. Also see above for detailed description on the first circumferential elements, second circumferential elements, connection elements, first strut, etc.

The amplitude of the first circumferential elements may range from about 0.5 mm to about 3 mm, from about 0.5 mm to about 2.5 mm, from about 0.5 mm to about 2 mm, from about 1 mm to about 2 mm, from about 1 mm to about 1.5 mm, or 1.47 mm. The amplitude of the second or third circumferential elements may range from about 0.2 mm to about 2 mm, from about 0.3 mm to about 1.5 mm, from about 0.3 mm to about 1 mm, from about 0.5 mm to about 1 mm, 0.81 mm or 0.83 mm.

Stents of the present invention may employ one, two or more end zones. The end zone may take numerous forms. An end zone may be formed from a plurality of cylindrical elements, and is connected to the main body at one or more bridging elements. Adjacent cylindrical elements may be connected directly, or may be connected by at least one second strut. In one embodiment, an end zone contains a first cylindrical element and a second cylindrical element. The first cylindrical element is substantially identical to the second cylindrical element except that it is rotated to have a different orientation.

The end zone may further comprise at least one radiopaque marker. See, www.nitinol-europe.com/pdfs/stentdesign.pdf for a review of the design and makeup of radiopaque markers which are well known in the art. The radiopaque markers may assume a variety of different sizes and shapes. For example, a radiopaque marker may contain a centrally placed marker hole. The marker hole may be circular or semicircular, but may also assume other shapes, such as a semicircular hole with an extrusion or dimple positioned at one portion of the circumference, or a hole in the shape of a heart.

The radiopaque marker may be electron-dense or x-ray refractile markers, such as metal particles or salts. Non-limiting examples of suitable marker metals include iron, gold, colloidal silver, zinc and magnesium, either in pure form or as organic compounds. Other radiopaque materials are tantalum, tungsten, platinum/iridium, or platinum. Heavy metal and heavy earth elements are useful in variety of compounds such as ferrous salts, organic iodine substances, bismuth or barium salts, etc. Further embodiments that may be utilized may encompass natural encapsulated iron particles such as ferritin that may be further cross-linked by cross-linking agents. Ferritin gel can be constituted by cross-linking with low concentrations (0.1-2%) of glutaraldehyde. The radiopaque marker may be constituted with a binding agent of one or more biodegradable polymer, such as PLLA, PDLA, PLGA, PEG, etc. In one embodiment comprising a radiopaque marker, iron containing compounds or iron particles are encapsulated in a PLA polymer matrix to produce a pasty substance, which can be injected or otherwise deposited in the hollow receptacle contained about the stent.

The stents may also have a transition zone between the end zone and the main body. The transition zone may be formed from a plurality of undulations, each undulation comprising two adjacent struts connected by a loop with the width of the loop varying across the transition zone. The transition zone may comprise a plurality of polygons where the surface area of adjacent polygons in the transition zone increases circumferentially. U.S. Patent Publication No. 20110125251. The transition zone may take other suitable configurations.

The dimensions of the stent may vary from about 10 mm to about 300mm in length, from 20 mm to about 300 mm in length, from about 40 mm to about 300 mm in length, from about 20 mm to about 200 mm in length, from about 60 mm to about 150 mm in length, or from about 80 mm to about 120 mm in length. In one embodiment, the stent may be about 88.9 mm. The internal diameter (I.D.) of the stent may range from about 2 mm to about 25 mm, from about 2 mm to about 5 mm (e.g., for the coronary arteries), from about 4 mm to about 8 mm (e.g., for neurological spaces in the CNS, both vascular and nonvascular), from about 6 mm to about 12 mm (e.g., for the iliofemoral), from about 10mm to about 20 mm (e.g., for the ilioaortic) and from about 10 mm to about 25 mm (e.g., for the aortic).

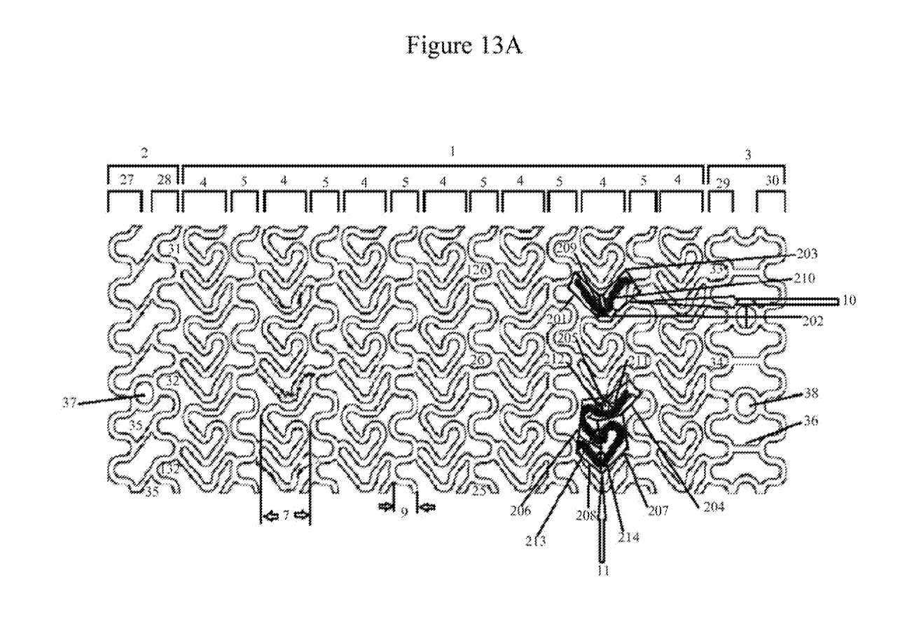

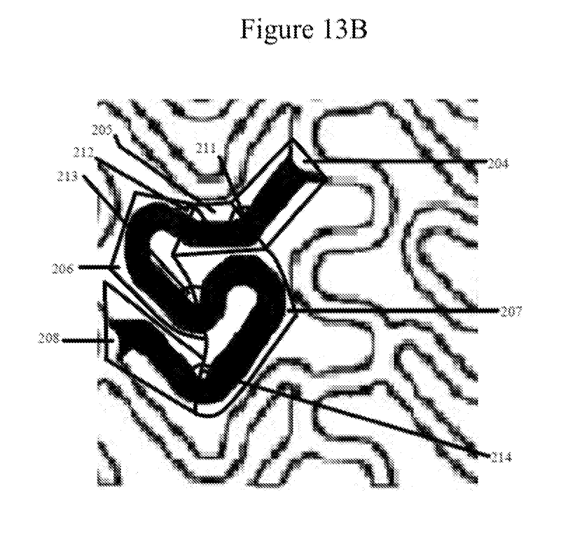

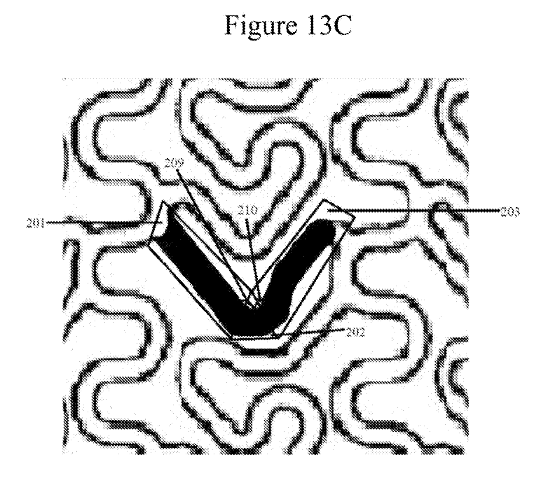

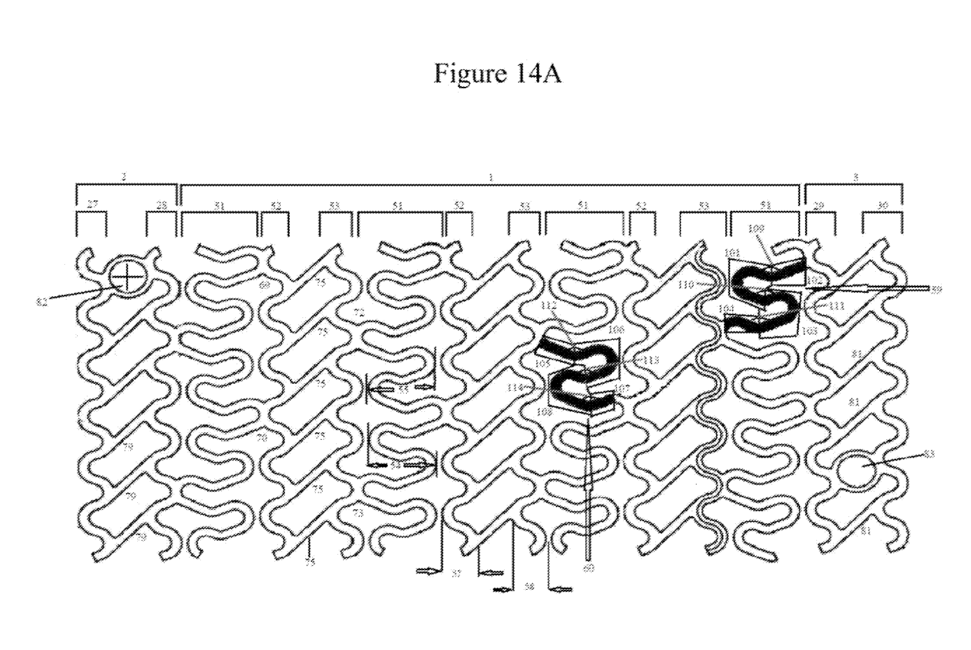

FIG. 13A is a planar view of one embodiment of the stent in an unexpanded state. As used herein, an unexpanded state can mean an as cut state or a crimped state. Although an unexpanded state can mean an as cut state or a crimped state, the diameter of the cross section of the as cut state of a stent may be greater than the diameter of the cross section of the crimped state. The stent comprises a main body 1 and two end zones 2, 3. The main body comprises a plurality of first circumferential elements 4 and a plurality of second circumferential elements 5 which are arranged in an alternating pattern. The circumference of the first circumferential element 4 is greater than the circumference of the second circumferential elements 5.

The first circumferential element 4 comprises a plurality of first undulations 10 and a plurality of second undulations 11 (hatched region. For clarity of illustration, only selected undulations are labeled, although there are more undulations). The first and second undulations are arranged in an alternating pattern in the first circumferential element 4. The undulation, 10, comprises three segments 201-203 (segments shown in boxed regions). The undulation, 11, comprises five segments 204-208. The segment contains both linear and curved portions that are joined together. When the stent is unexpanded, the segments, 201-203, form angles 209, 210. Similarly, segments 204-208 form angles 211-214 (note for the sake of clarity, only a selected group of angles are illustrated). The amplitude of the second undulations 11 is shown as 7.

The second circumferential element 5 contains a plurality of undulations forming a sinusoidal pattern (FIGS. 13A-C). When expanded, the second circumferential element forms a hoop-like structure where the hoop is substantially in the same plane. In the second circumferential elements, each peak is axially spaced a similar distance from each valley such that the undulations in the second circumferential elements share a constant amplitude 9. The amplitude 7 of the undulation 11 in the first circumferential element 4 is greater than the amplitude 9 of the undulations in the second circumferential elements 5.

The first circumferential element 4 is connected to an adjacent second circumferential element 5 by connection elements, 25, 26, 126, which is an adjoining region of the first circumferential clement 4 and second circumferential clement 5. In this case, adjacent first circumferential element 4 and second circumferential element 5 are directly connected, forming a four-way junction. The connection elements 25, 26, 126, are positioned symmetrically; so the radial distance between each pair of connection elements is equal. Because there are three equally spaced connection elements 25, 26,126, between adjacent circumferential elements 4, 5, they are situated approximately 120° with respect to one another.

The stent in FIG. 13A also employs two end zones 2, 3. The end zone 2 (or 3) is formed from cylindrical elements 27, 28 (or 29, 30 for end zone 3), and is connected to the main body at bridging elements 31, 32, 132 (or 33, 34, 134 for end zone 3). The cylindrical elements are connected by a plurality of second struts 35 (or 36 for end zone 3; note for clarity, only a selected group of second struts are labeled). The end zones 2, 3, further comprise radiopaque markers 37 for end zone 2, and 38 for end zone 3. The radiopaque markers 37, 38, contain a centrally placed marker hole.

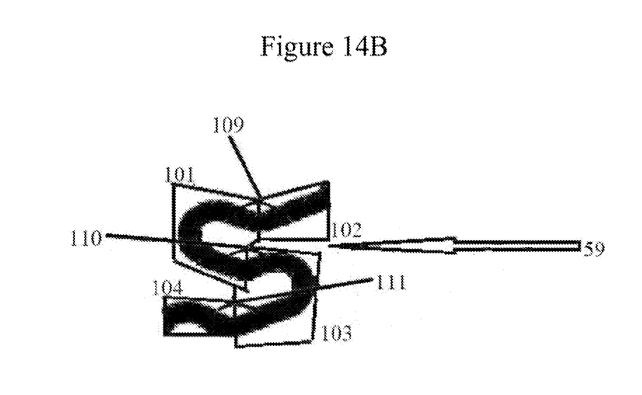

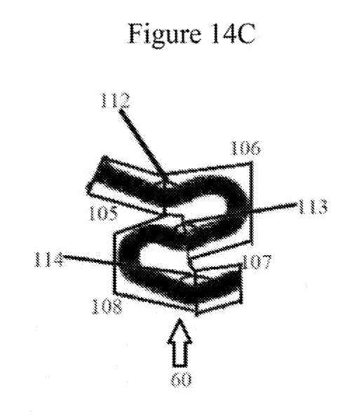

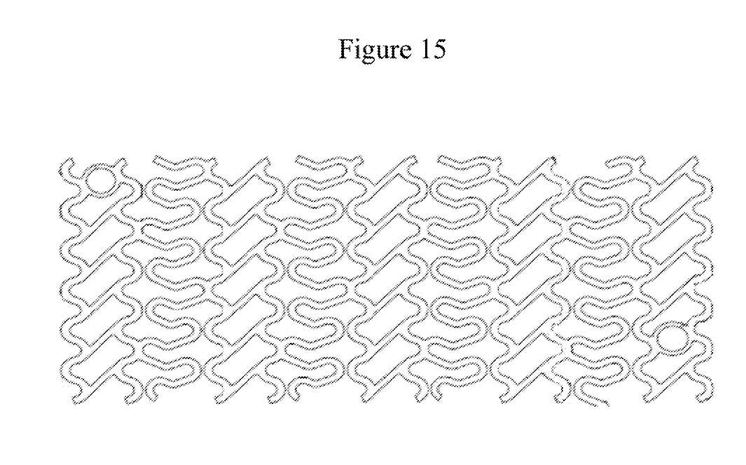

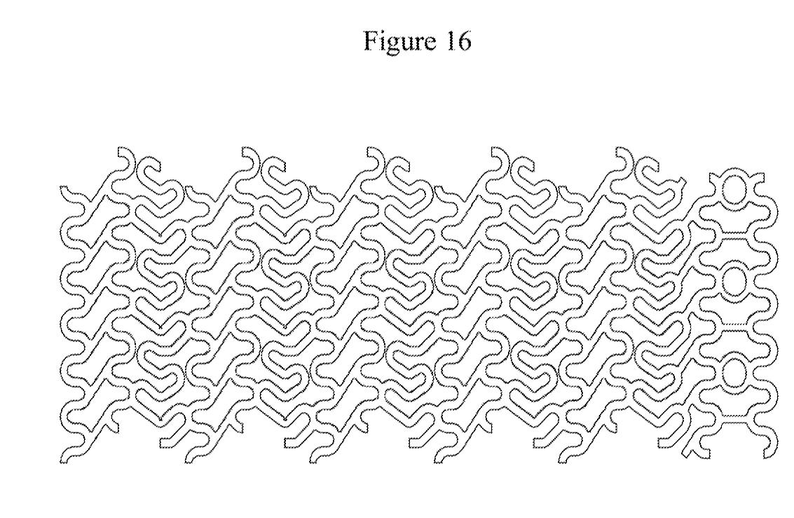

FIG. 14A is a planar view of one embodiment of the stent in an unexpanded state. The stent comprises a main body 1 and two end zones, 2, 3. The main body comprises a plurality of first circumferential elements 51, a plurality of second circumferential elements 52 and a plurality of third circumferential elements 53. The first, 51, second, 52 and third, 53 circumferential elements are arranged in a repeating pattern where the main body comprises four repeats of first, second and third circumferential elements (i.e., a first circumferential element 51 followed by a second circumferential element 52 which is again followed by a third circumferential element 53). Two first circumferential elements 51 are positioned at both ends of the repeating blocks and connect directly with the end zones 2 and 3, respectively. The circumference of the first circumferential element 51 is greater than the circumference of the second, 52, and third, 53, circumferential elements.

The first circumferential element 51 comprises a plurality of first undulations 59 and a plurality of second undulations 60 (hatched region. For clarity of illustration, only selected undulations are labeled, although there are more undulations). The first, 59, and second, 60, undulations are arranged in an alternating pattern in the first circumferential element 51. Each undulation, 59, 60, comprises four angled segments 101-104 (undulation 59; segments shown in boxed regions), or 105-108 (undulation 60). The segment contains both linear and curved portions that arc joined together. When the stent is crimped, the segments, 101-104, form angles 109-111. Similarly, segments 105-108 form angles 112-114 (note for the sake of clarity, only a selected group of angles are illustrated).

The second circumferential element 52 contains a plurality of undulations forming a sinusoidal pattern (FIGS. 14A-C). The third circumferential element 53 contains a plurality of undulations forming a sinusoidal pattern (FIGS. 14A-C). When expanded, the second and third circumferential elements form a hoop-like structure where the hoop is substantially in the same plane. The amplitude of the first undulation 59 is shown as 54. The amplitude of the second undulation 60 is shown as 55. In the second circumferential elements 52, each peak is axially spaced a similar distance from each valley such that undulations in the second circumferential element 52 share a constant amplitude 57. In the third circumferential elements 53, each peak is axially spaced a similar distance from each valley such that undulations in the third circumferential element 53 share a constant amplitude 58. The amplitude 54 (or 55) of the undulations in the first circumferential element 51 is greater than the amplitude 57 of the undulations in the second circumferential elements 52, and is greater than the amplitude 58 of the undulations in the third circumferential elements 53.

The first circumferential element 51 is connected to an adjacent second circumferential element 52 by the connection elements, 69, 70, which are adjoining regions of adjacent first circumferential element and second circumferential element. In this case, adjacent first circumferential element and second circumferential element are directly connected, forming a four-way junction. The connection elements 69, 70, are positioned symmetrically; so the radial distance between each pair of connection elements is equal. When there are two equally spaced connection elements 69, 70, between adjacent first and second circumferential elements 51, 52, they are situated approximately 180° with respect to one another.

The first circumferential element 51 is connected to an adjacent third circumferential element 53 by the connection elements, 72, 73, which are adjoining regions of adjacent first circumferential element and third circumferential element. In this case, adjacent first circumferential element and third circumferential element are directly connected, forming a four-way junction. The connection elements 72, 73, are positioned symmetrically; so the radial distance between each pair of connection elements is equal. When there are two equally spaced connection elements 72, 73, between adjacent first and third circumferential elements 51, 53, they arc situated approximately 180° with respect to one another.

The second circumferential element 52 is connected to an adjacent third circumferential element 53 by first struts, 75 (for clarity, only selected groups of first struts are labeled, although there are more first struts).

The stent in FIG. 14A also employs two end zones 2, 3. The end zones 2, 3, are formed from cylindrical elements 27, 28 (for end zone 2), 29, 30 (for end zone 3), and are directly connected to the first circumferential elements 51 of the main body 1. The cylindrical elements 27, 28 are connected by a plurality of second struts 79. The cylindrical elements 29, 30 are connected by a plurality of third struts 81 (note for clarity, only a selected group of second and third struts are labeled). The end zones, 2, 3, further comprises radiopaque markers 82, 83. The radiopaque markers, 82, 83, contain a centrally placed marker hole.

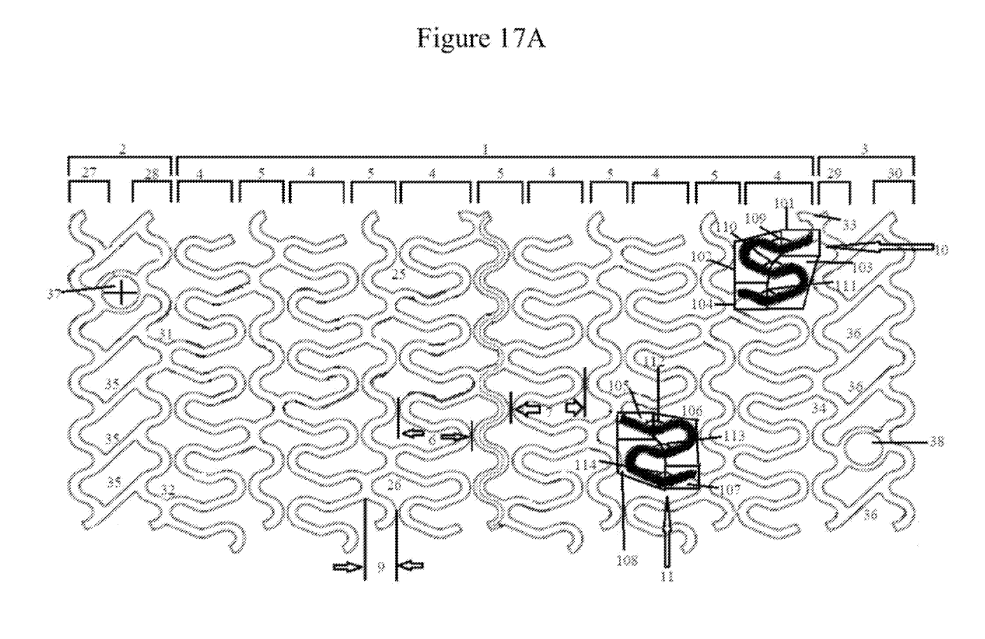

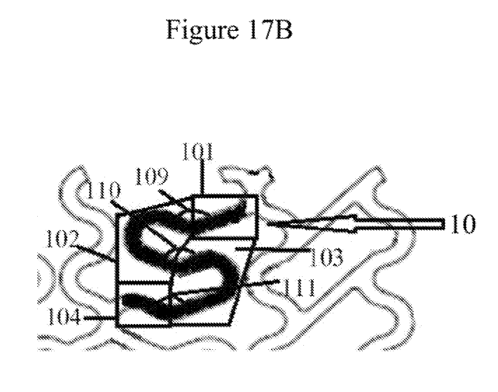

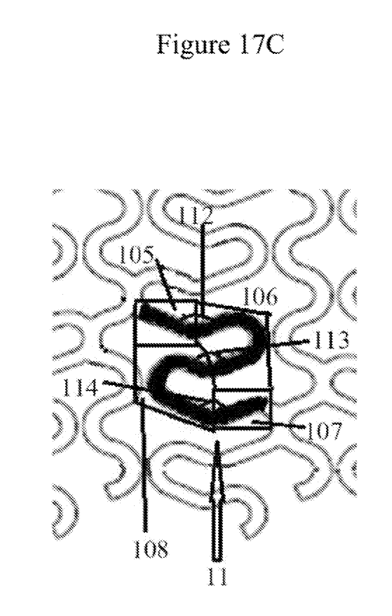

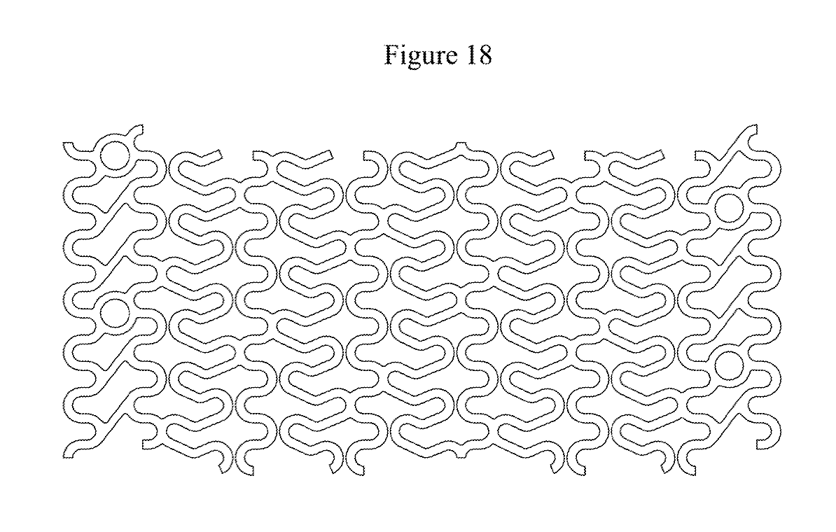

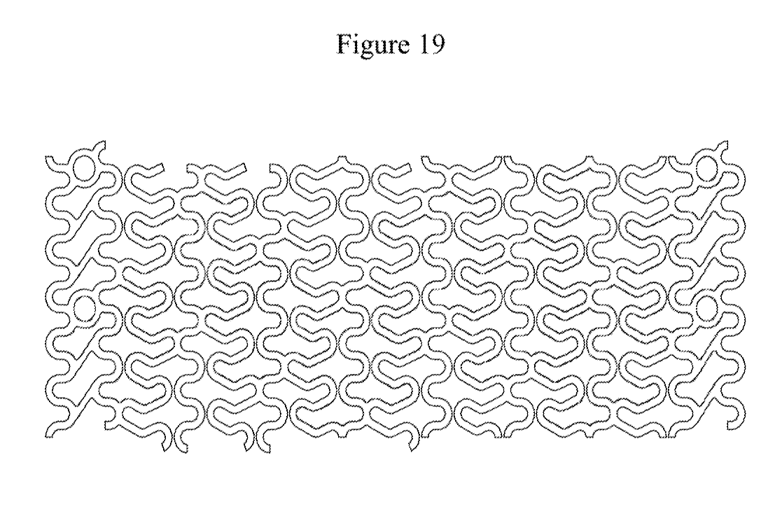

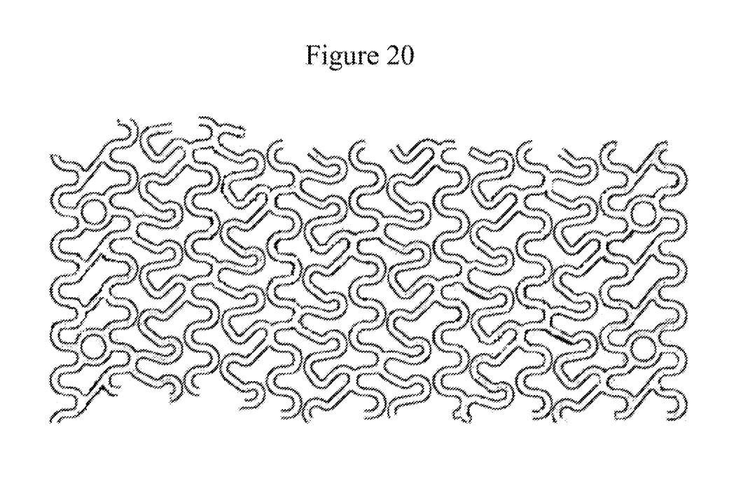

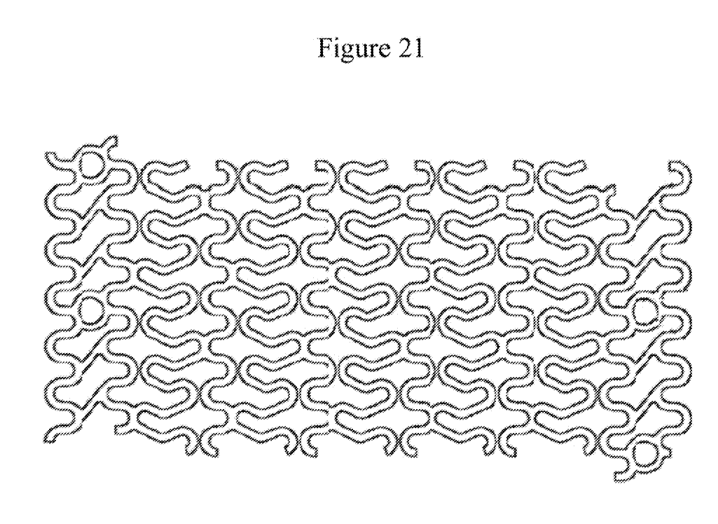

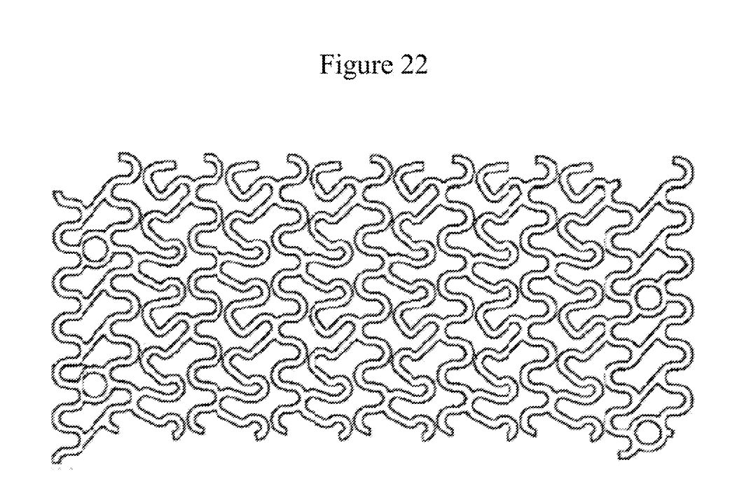

FIG. 17A is a planar view of one embodiment of the stent in an unexpanded state. The stent comprises a main body 1 and two end zones 2, 3. The main body comprises a plurality of first circumferential elements 4 and a plurality of second circumferential elements 5 which are arranged in an alternating pattern. The circumference of the first circumferential element 4 is greater than the circumference of the second circumferential elements 5.

The first circumferential element 4 comprises a plurality of first undulations 10, and a plurality of second undulations 11 (hatched region. For clarity of illustration, only selected undulations are labeled, although there are more undulations). The first, 10, and second, 11, undulations are arranged in a repeating pattern in the first circumferential element 4. Each undulation, 10 or 11, comprises four segments 101-104 (undulation 10; segments shown in boxed regions), 105-108 (undulation 11). The segment contains both linear and curved portions that are joined together. When the stent is unexpanded, the segments, 101-104, form angles 109-111. Similarly, segments 105-108 form angles 112-114 (note for the sake of clarity, only a selected group of angles are illustrated). The amplitude of the first undulation 10, and the second undulation 11 is shown as 6, 7, respectively.

The second circumferential element 5 contains a plurality of undulations forming a sinusoidal pattern (FIGS. 17A-C). When expanded, the second circumferential element forms a hoop-like structure where the hoop is substantially in the same plane. In the second circumferential elements, each peak is axially spaced a similar distance from each valley such that undulations in the second circumferential clement share a constant amplitude 9. The amplitude 6 (or 7) of the undulations in the first circumferential element 4 is greater than the amplitude 9 of the undulations in the second circumferential elements 5.

The first circumferential element 4 is connected to an adjacent second circumferential element 5 by connection elements, 25, 26, which is an adjoining region of the first circumferential element and second circumferential element. In this case, adjacent first circumferential element and second circumferential element are directly connected, forming a four-way junction. The connection elements 25, 26, are positioned symmetrically; so the radial distance between each pair of connection elements is equal. When there are two equally spaced connection elements 25, 26, between adjacent circumferential elements 4, 5, they are situated approximately 180° with respect to one another. In other words, the two connection elements, 25, 26, are oppositely oriented with respect to one another.

The stent in FIG. 17A also employs two end zones 2, 3. The end zone 2 (or 3) is formed from cylindrical elements 27, 28 (or 29, 30 for end zone 3), and is directly connected to the main body at bridging elements 31, 32 (or 33, 34 for end zone 3). The cylindrical elements are connected by a plurality of second struts 35 (or 36 for end zone 3; note for clarity, only a selected group of second struts are labeled). The end zones 2, 3, further comprise radiopaque markers 37 for end zone 2, and 38 for end zone 3. The radiopaque markers 37, 38, contain a centrally placed marker hole.

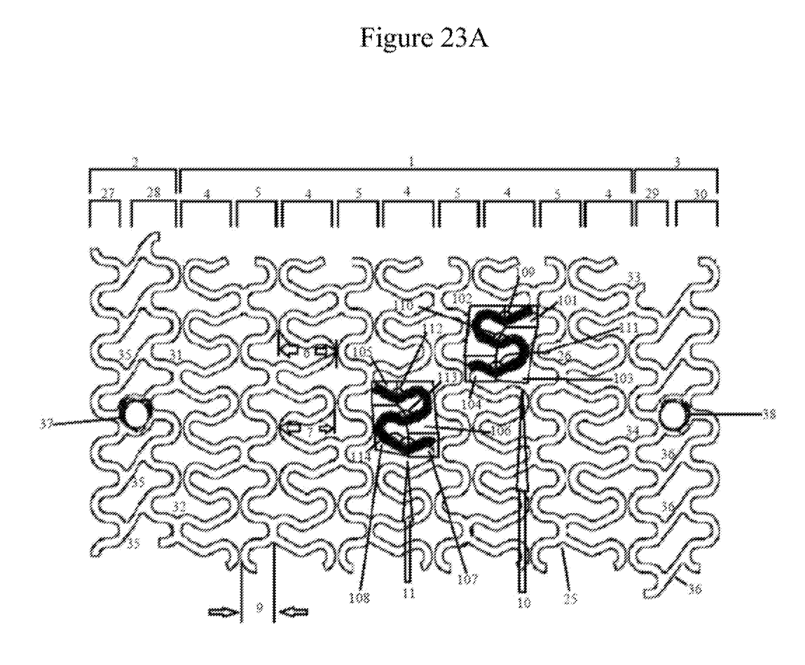

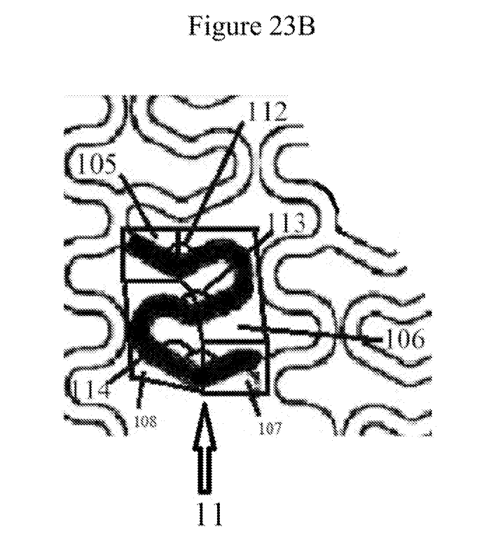

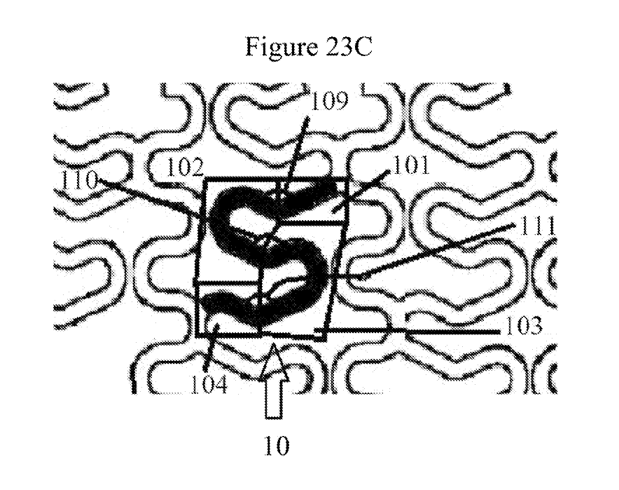

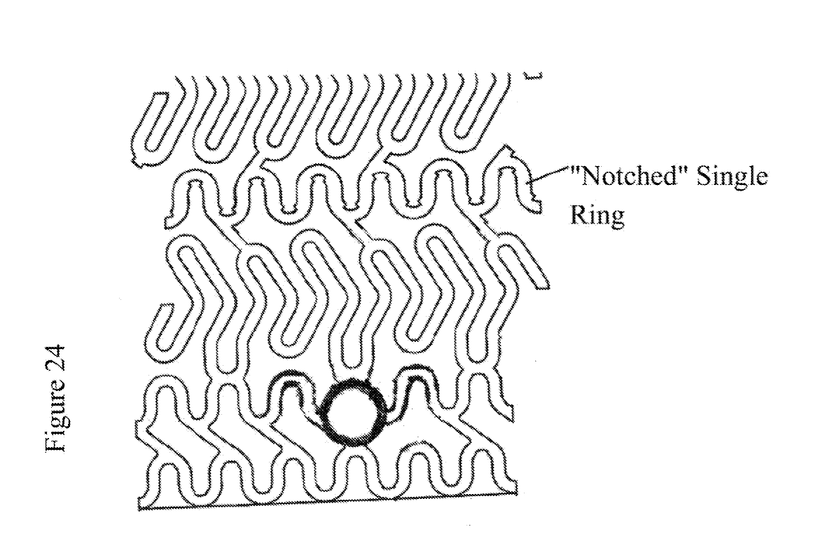

FIG. 23A is a planar view of one embodiment of the stent in an unexpanded state. The stent comprises a main body 1 and two end zones 2, 3. The main body comprises a plurality of first circumferential elements 4 and a plurality of second circumferential elements 5 which are arranged in an alternating pattern. The circumference of the first circumferential element 4 is greater than the circumference of the second circumferential elements 5.

The first circumferential element 4 comprises a plurality of first undulations 10, and a plurality of second undulations 11 (hatched region. For clarity of illustration, only selected undulations are labeled, although there are more undulations). The first, 10, and second, 11 undulations are arranged in a repeating pattern in the first circumferential element 4. Each undulation, 10 or 11, comprises four segments 101-104 (undulation 10; segments shown in boxed regions), 105-108 (undulation 11). The segment contains both linear and curved portions that arc joined together. When the stent is unexpanded, the segments, 101-104, form angles 109-111. Similarly, segments 105-108 form angles 112-114 (note for the sake of clarity, only a selected group of angles are illustrated). The amplitude of the first undulation 10, and second undulation 11, is shown as 6 and 7, respectively.

The second circumferential element 5 contains a plurality of undulations forming a sinusoidal pattern (FIGS. 23A-C). When expanded, the second circumferential element forms a hoop-like structure where the hoop is substantially in the same plane. In the second circumferential elements, each peak is axially spaced a similar distance from each valley such that undulations in the second circumferential element share a constant amplitude 9. The amplitude 6 (or 7) of the undulations in the first circumferential element 4 is greater than the amplitude 9 of the undulations in the second circumferential elements 5.

The first circumferential element 4 is connected to an adjacent second circumferential element 5 by at least one connection element, 25, 26, which is an adjoining region of the first circumferential element and second circumferential element. In this case, adjacent first circumferential element and second circumferential element are directly connected, forming a four-way junction. The connection elements 25, 26, are positioned symmetrically; so the radial distance between each pair of connection elements is equal. When there are two equally spaced connection elements 25, 26, between adjacent circumferential elements 4, 5, they are situated approximately 180° with respect to one another. In other words, the two connection elements, 25, 26, are oppositely oriented with respect to one another.

The stent in FIG. 23A also employs two end zones 2, 3. The end zone 2 (or 3) is formed from cylindrical elements 27, 28 (or 29, 30 for end zone 3), and is connected to the main body at bridging elements 31, 32 (or 33, 34 for end zone 3). The cylindrical elements are connected by a plurality of second struts 35 (or 36 for end zone 3; note for clarity, only a selected group of second struts are labeled). The end zones 2, 3, further comprise radiopaque markers 37 for end zone 2, and 38 for end zone 3. The radiopaque markers 37, 38, contain a centrally placed marker hole.

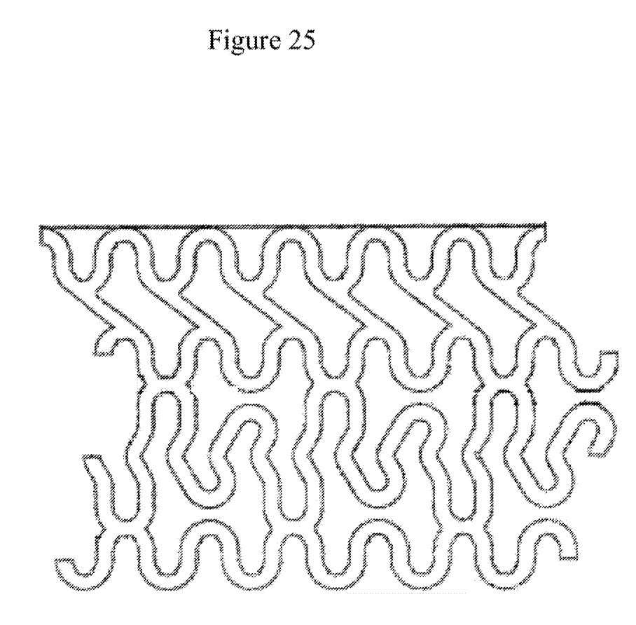

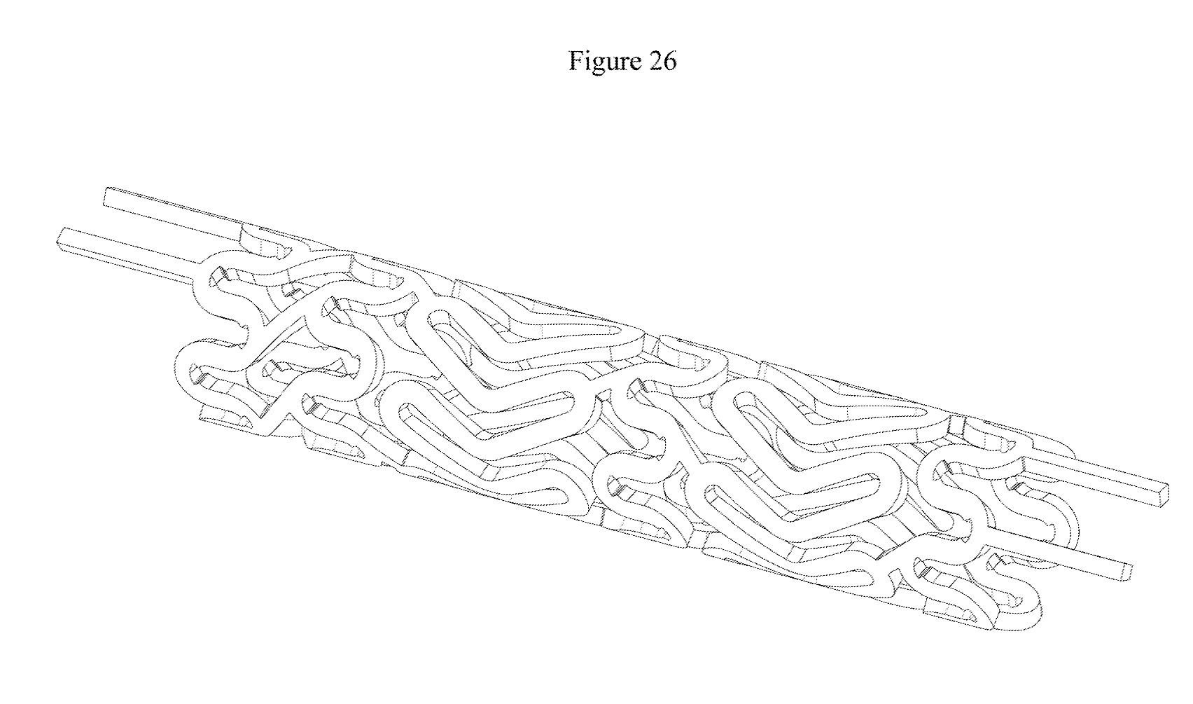

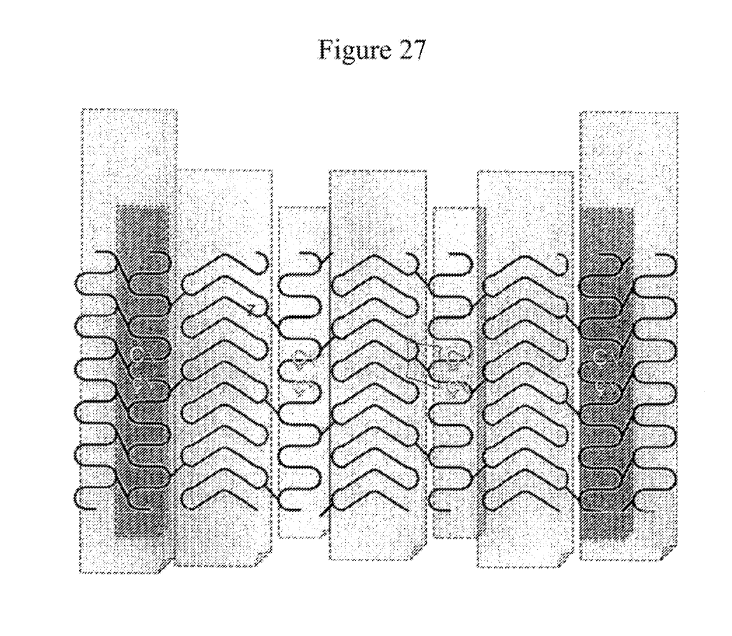

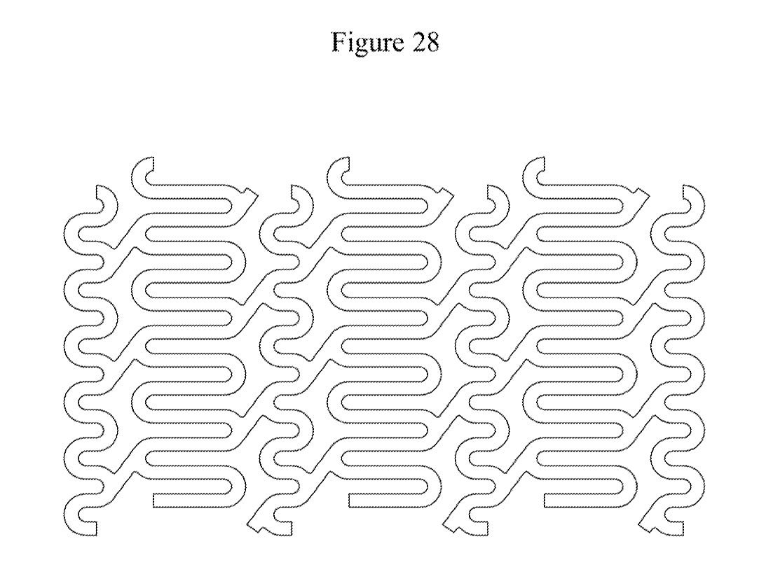

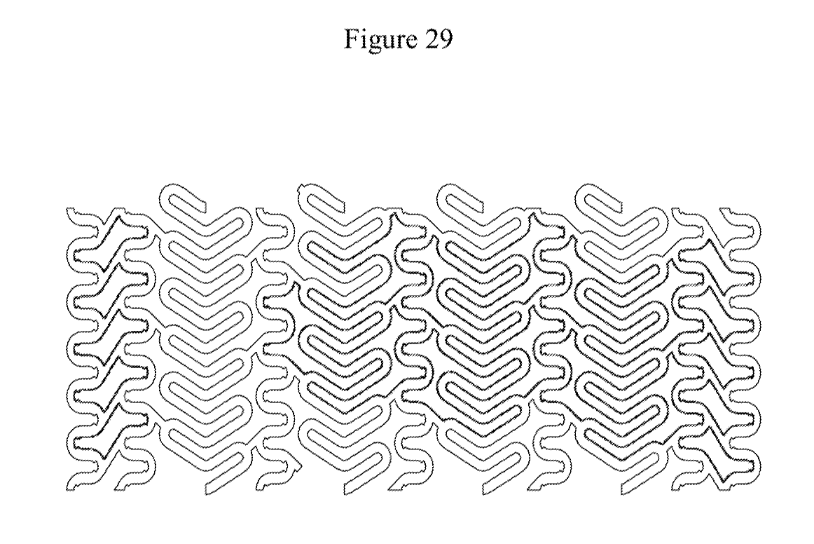

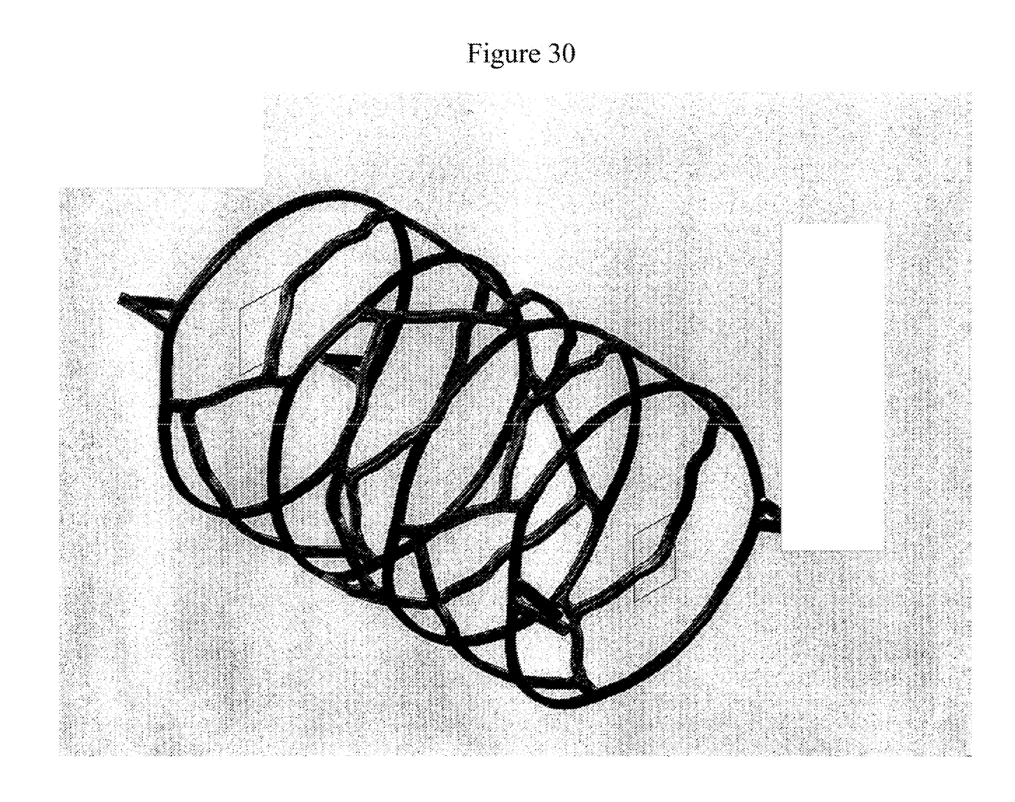

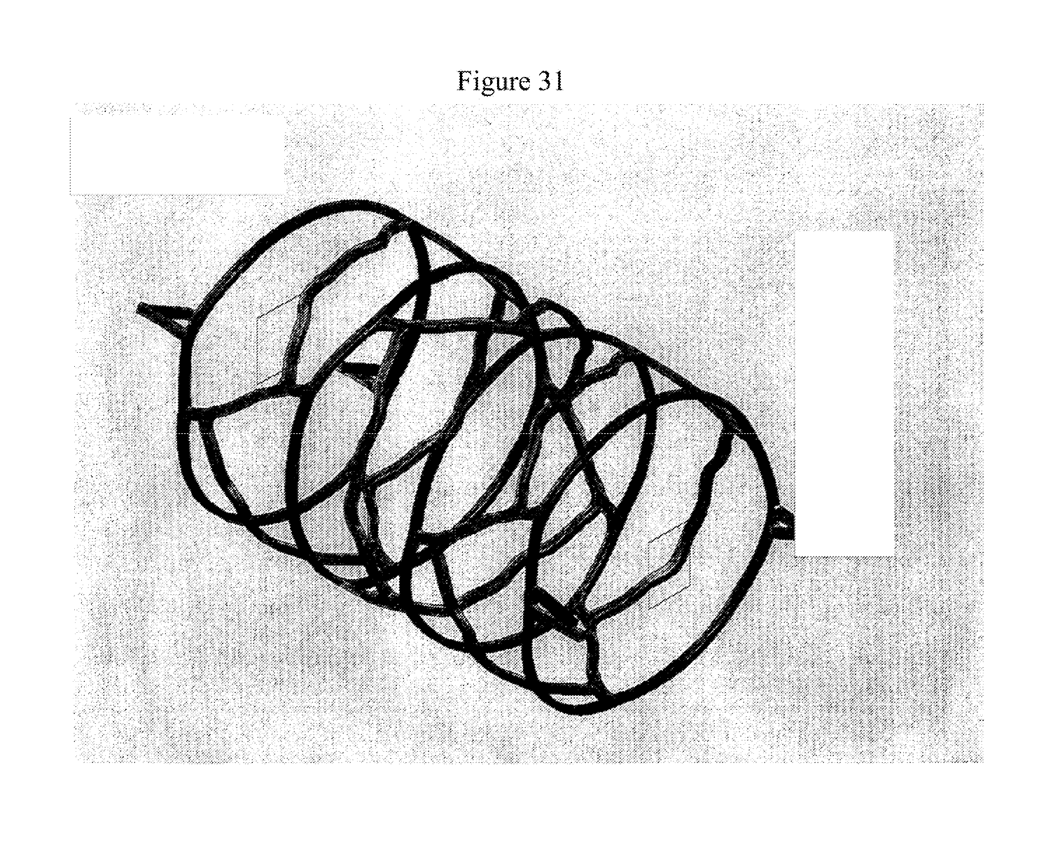

FIG. 24 shows a portion of a stent where a second circumferential element comprises notches, 201-211, at the peak and valley regions of the undulations. The notches can also be other types of indentations. The indentation(s) can be at any points/regions of the second circumferential element. When the stent expands, the second circumferential element expands circumferentially to form a ring or hoop structure. The notches (or other suitable indentations) help distribute the expansion stress uniformly along the second circumferential element, over a greater length, by, for example, increasing the number of focal points. Any suitable indentations can be used in the present stents, such as a dimple, a rounded indentation, an angular cut, an n-shaped indentation, etc. The number of the indentations on a second circumferential element can vary from 0 to 24, from 2 to 12, from 3 to 12, from 6 to 12. Higher numbers of indentations are also encompassed by the present invention.

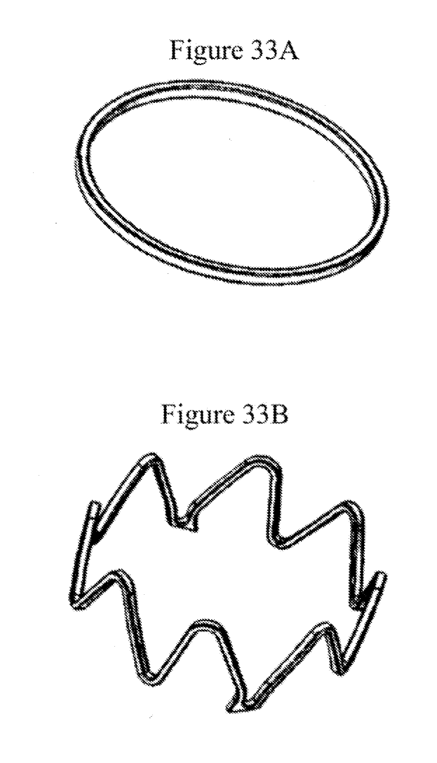

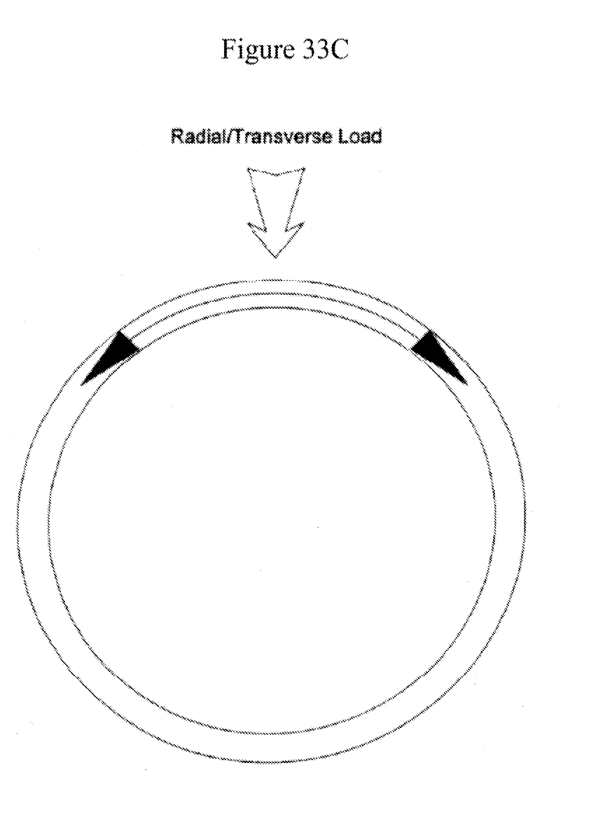

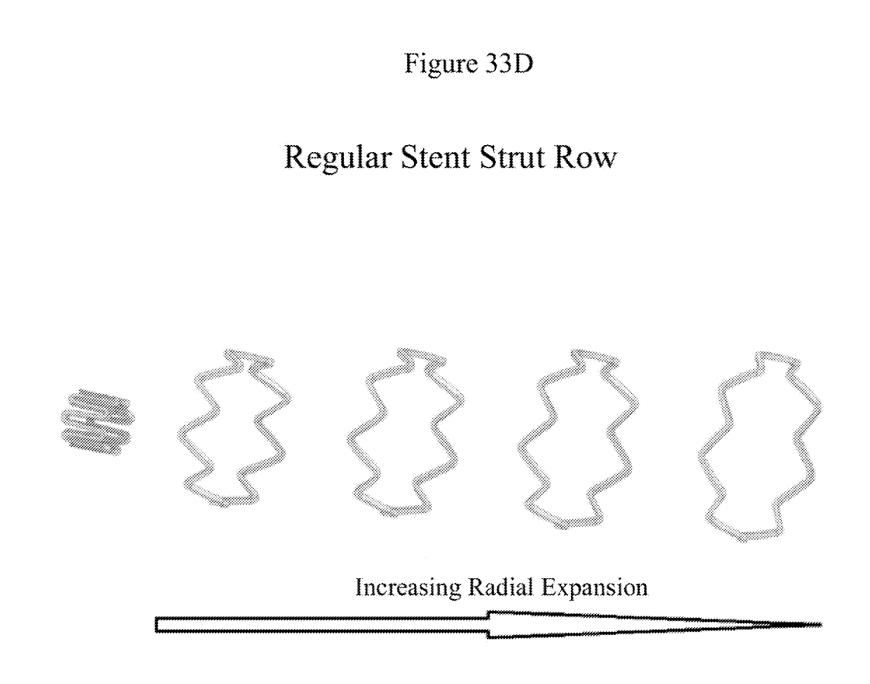

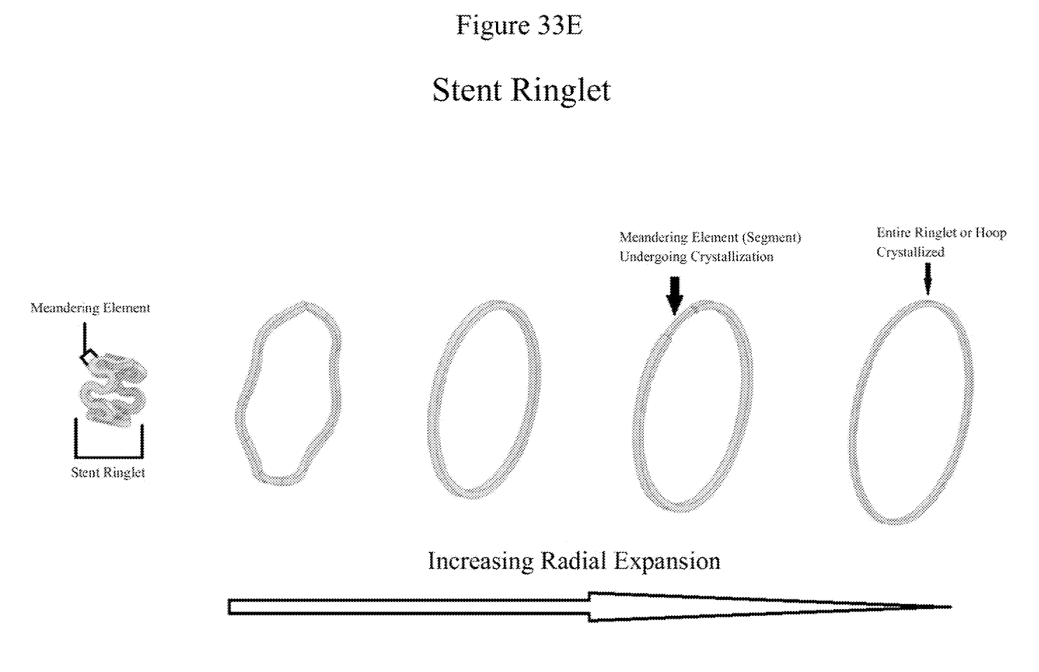

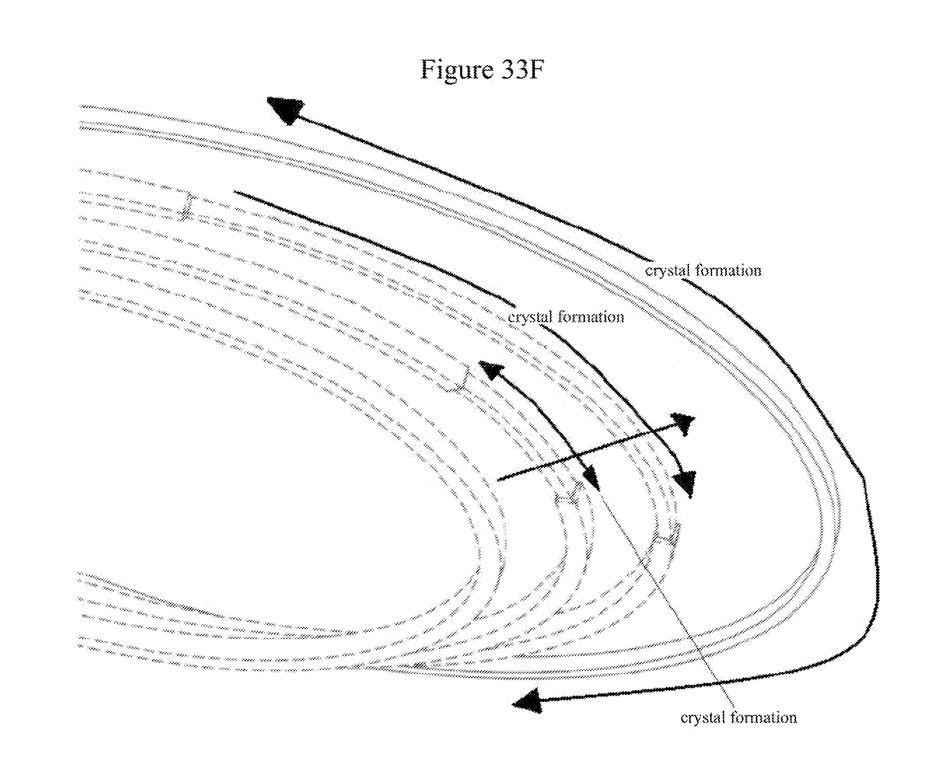

The stent of the present application can comprise at least one bioabsorbable polymer. The bioabsorbable polymer may be crystallizable. In certain embodiments, the second (or third) circumferential element may form a hoop or ring when expanded. FIG. 33A depicts a partial top view of an expanded hoop or ring, while FIG. 33B illustrates such hoop or ring when not expanded, shown in the drawing as composed of meandering sinusoidal (33B) bioabsorbable selements of a stent embodiment. FIG. 33C illustrates a hoop or ring element of a bioabsorbable stent showing how radial/transverse load is distributed through a ring structure, enabling the ring structure to expand circumferentially. As illustrated such structure provides a better distribution of forces keeping such stent open under forces that might otherwise cause deformation of the stent. FIG. 33D illustrates a hoop undergoing progressive radial expansion. FIG. 33E shows the stent ring undergoing increasing radial expansion. The circumferential element straightens and then undergoes deformation. The modulus of stretching could range from about 250,000 PSI to about 550,000 PSI. Deformation includes a decrease in the cross sectional dimension of one segment of the circumferential element (the width and thickness). One segment of the ring may undergo deformation with subsequent change in crystallization and/or decrease in the cross sectional area. In one embodiment, the cross sectional area decreases without any accompanying change in the crystal structure. During radial expansion, the number of segments of the circumferential elements undergoing such crystal formation and decrease in cross sectional area increases from 1, 2, 3 to n until the entire circumferential element or stent ring (hoop) has undergone such transformation. This phenomena, which can also be referred to as “necking” as the cross section of the ringlet decreases in a specific section of the circumferential element and crystallization spreads laterally around the ring. FIG. 33F. “The necking phenomena in polymers is well known and usually occurs when a homogeneous solid polymeric bar (film or filament), with a non-monotonous dependence of axial force S on stretching ratio λ, is stretched uniaxially . . . In this case the polymer bar is not deformed homogeneously. Instead, two almost uniform sections occur in the sample: one being nearly equal to its initial thickness and another being considerably thinner in the cross-sectional dimensions.” See, for example, Leonov, A. I., A Theory of Necking in Semi-Crystalline Polymers, Int'l J. of Solids and structures, 39 (2002)5913-5916; see also, http://www.eng.uc.edu/˜gbeaucag/ Classes/Characterization/StressStrainhtml/ StressStrain.html (May 6, 2010); see also, http://materials.npl.co.uk/NewIOP/ Polymer.html (May 6, 2010).

The device of the present invention may be used as a self-expanding stent or with any balloon catheter stent delivery system, including balloon catheter stent delivery systems described in U.S. Pat. Nos. 6,168,617, 6,222,097, 6,331,186 and 6,478,814. In one embodiment, the present device is used with the balloon catheter system disclosed in U.S. Pat. No. 7,169,162.

The device of the present invention may be used with any suitable catheter, the diameter of which may range from about 0.8mm to about 5.5 mm, from about 1.0 mm to about 4.5 mm, from about 1.2 mm to about 2.2 mm, or from about 1.8 to about 3 mm. In one embodiment, the catheter is about 6 French (2 mm) in diameter. In another embodiment, the catheter is about 5 French (1.7 mm) diameter.

The stent may be inserted into the lumen of any vessel or body cavity expanding its cross-sectional lumen. The invention may be deployed in any artery, vein, duct or other vessel such as a ureter or urethra and may be used to treat narrowing or stenosis of any artery, including, the coronary, infrainguinal, aortoiliac, subclavian, mesenteric or renal arteries. Other types of vessel obstructions, such as those resulting from a dissecting aneurysm are also encompassed by the invention.

The subjects that can be treated using the stent and methods of this invention are mammals, including a human, horse, dog, cat, pig, rabbit, rodent, monkey and the like.

The stent of the present invention may be formed from at least one bioabsorbable polymer representing a wide range of different polymers. Typically, bioabsorbable polymers comprise aliphatic polyesters based on lactide backbone such as poly L-lactide, poly D-lactide, poly D,L-lactide, mesolactide, glycolides, lactones, as homopolymers or copolymers, as well as formed in copolymer moieties with co-monomers such as, trimethylene carbonate (TMC) or ε-caprolactone (ECL). U.S. Pat. Nos. 6,706,854 and 6,607,548; EP 0401844; and Jeon et al. Synthesis and Characterization of Poly (L-lactide)—Poly (ε-caprolactone). Multiblock Copolymers Macromolecules 2003: 36, 5585-5592. The copolymers comprises a moiety such as L-lactide or D-lactide of sufficient length that the copolymer can crystallize and not be sterically hindered by the presence of glycolide, polyethylene glycol (PEG), ε-caprolactone, trimethylene carbonate or monomethoxy-terminated PEG (PEG-MME). For example, in certain embodiments greater than 10, 100 or 250 L or D-lactides may be arrayed sequentially in a polymer. The stent may also be composed of bioabsorbable polymeric compositions such as those disclosed in U.S. Pat. No. 7,846,361 and applicant's co-pending U.S. Patent Publication No. 2010/0093946.

The following nomenclature will now be used with the polymer nomenclature being based on the presence of the monomer type.

| | |
|---|---|
| LPLA: | Poly(L-lactide) |
| LPLA-PEG: | Poly(poly-L-lactide-polyethylene glycol) |
| DPLA: | Poly(D-lactide) |
| DPLA-TMC: | Poly(poly D-lactide-co-trimethylene carbonate) |
| DLPLA: | Poly(DL-lactide), a racemic copolymer D-co-L-lactide |
| LDPLA: | Poly(L-co-D-lactide) |
| LDLPLA: | Poly(L-co-DL-lactide), named for the method of monomer |
| | introduction |
| PGA: | Poly(glycolide) |
| PDO: | Poly(dioxanone) (PDS is Trademark) |
| SR: | “Self reinforced” (a processing technique) |
| TMC: | Trimethylene carbonate |
| PCL: | Poly(ε-caprolactone) |
| LPLA-TMC: | Poly(poly L-lactide-co-trimethylene carbonate) |
| LPLG: | Poly(L-lactide-co-glycolide) |
| POE: | Poly Orthoester |

In an embodiment of the present invention, the composition comprises a base polymer of poly(L-lactide) or poly(D-lactide). Advantageous base polymer compositions include blends of poly(L-lactide) and poly(D-lactide). Other advantageous base polymer compositions include poly(L-lactide-co-D,L-lactide) or poly(D-lactide-co-D,L-lactide) with a D,L-lactide co-monomer molar ratio from 10 to 30%, and poly(L-lactide-co-glycolide) or poly(D-lactide-co-glycolide) with a glycolide co-monomer molar ratio from 10 to 20%.

Another embodiment embodies a base polymer featuring a poly (L-lactide) moiety, and/or a poly (D-lactide) moiety, linked with a modifying copolymer thereof, including poly (L-lactide-co-tri-methylene-carbonate or poly(D-lactide-co-tri-methylene-carbonate) and (L-lactide-co-.epsilon.-caprolactone), or poly(D-lactide-co-.epsilon.-caprolactone), in the form of block copolymers or blocky random copolymers, wherein the lactide chain length is sufficient to affect cross-moiety crystallization.

In another embodiment, the polymer composition allows the development of the lactide racemate (stereo complex) crystal structure, between the L and D moieties, to further enhance the mechanical properties of the bioabsorbable polymer medical device. The formation of the racemate (stereo complex) crystal structure can accrue from formulations such as combinations of:
Poly L-lactide with Poly D-lactide with Poly L-lactide-co-TMC;
Poly D-lactide with Poly L-lactide-co-TMC;
Poly L-lactide with Poly D-lactide-co-TMC;
Poly L-lactide with Poly D-lactide with Poly D-lactide-co-TMC;
Poly L-lactide-co-PEG with Poly D-lactide-co-TMC; and
Poly D-lactide-co-PEG with Poly L-lactide-co-TMC.

Poly-lactide racemate compositions of this embodiment may have an especially advantageous characteristic in being “cold formable or bendable” without adding heat. Cold-bendable scaffolds of the invention do not require beating to become flexible enough to be crimped onto a carrier device or accommodate irregularly shaped organ spaces. Cold bendable ambient temperatures are defined as room temperature not exceeding 30.degree. C. Cold-bendable scaffolds, for example, afford sufficient flexibility when implanted allowing for an expanded scaffold device in an organ space such as pulsating vascular lumen. For example, in terms of a stent, it may be desirable to utilize polymeric compositions that afford mostly amorphous polymer moieties after fabrication that can crystallize particularly when the secondary nested or end-positioned meandering struts when the scaffold is strained by stretching upon balloon expansion for implantation. Such cold-bendable polymeric scaffold embodiments of are not brittle and do not have to be preheated to a flexible state prior to implantation onto a contoured surface space in the body. Cold-bendability allows these blends to be crimped at room temperature without crazing, and moreover, the blends can be expanded at physiological conditions without crazing.

Poly-lactide racemate compositions and non-racemate compositions of embodiments herein may be processed to have blocky moieties allowing cross moiety crystallization even with the addition of an impact modifier to the blend composition. Such a blend introduces the possibility to design device specific polymer compositions or blends by producing either single or double Tg's (glass melt transition points).

Poly-lactide racemate compositions may show significant improvement in re-crystallization capability over, for example, non-racemate PLDL-lactide blends. An advantageous racemate alignment of the different polylactide moieties can be achieved, for example, by blending a poly-D-lactide with the copolymer poly L-lactide-co-TMC capable of forming racemate crystal across the different polylactide stereomoieties, for example, without limitation, when stretched during expansion to the required emplacement diameter. This strain induced crystallization, without adverse crazing, results in an increase of the mechanical properties reflected also in a positive change of modulus data over the base of the base materials.

Cross moiety crystallization of compositions with copolymers appears to be limited to copolymer with monomer molar ratios ranging from about 90:10 through 50:50. In fact, at a molar ratio of 50:50, the polymer moieties sterically impeded crystallization whereas the greater ratios are much more suitable for cross moiety crystallization. On the basis of experimental induced crystallization, different blends with various concentrations of lactide copolymers such as TMC or .epsilon.CL, to which an excess of poly (D-lactide) for racemate alignment with the L-lactide component has been added, the effective concentration of the copolymer in a racemate composition may be equal to, or less than, 40%. Thus, the thermal cross-links formed by cross moiety crystallization serves to reduce elongation or creep while maintaining the intended toughening mechanism. The advantageously strong racemate composition affords increased modulus data in tensile tests avoiding the method for reducing the tensile strength in the polymer blend.

An advantageous racemate composition embodiment provides a bioabsorbable polymer with minimal degradation in terms of high residual monomer level such that the contaminant monomeric residual fraction does not exceed about 0.5%, or preferably not in excess of about 0.3%. In embodiment concentration of monomeric contaminant of the polymer of the present invention is as low as about 0.2%.

Polymer compositions of embodiments described herein may comprise a base polymer present from about 70% to 95% by weight, or from about 70% to 80% by weight of the composition. For example, in one embodiment, the polymer formulation may comprise from about 70% by weight poly L-lactide (about 2.5 to 3 IV) with the poly L-lactide-co-TMC (70/30 mole/mole) (1.4 to 1.6 IV). In another embodiment, the polymer formulation may comprise 70% by weight triblock poly L-lactide-co-PEG (99/01 mole/mole) (2.5 to 3 IV) with the poly L-lactide-co-TMC (70/30 mole/mole) (1.4 to 1.6 IV). Furthermore, the polymer composition may comprise a formulation of about 70% by weight diblock poly L-lactide-co-PEG-MME (95/05 mole/mole) (2.5 to 3 IV) with poly L-lactide-co-TMC (70/30 mole/mole) (1.4 to 1.6 IV). Other embodiments provide formulations wherein .epsilon.-caprolactone is substituted in a composition for the aforementioned TMC. Similarly, an embodiment may provide formulations wherein PEG-MME may be substituted for PEG.

As is understood in this art, polymer compositions of the present invention can be customized to accommodate various requirements of the selected medical device. The requirements include mechanical strength, elasticity, flexibility, resilience, and rate of degradation under physiological and localized anatomical conditions. Additional effects of a specific composition concern solubility of metabolites, hydrophilicity and uptake of water and any release rates of matrix attached or enclosed pharmaceuticals.

The polymer implant utility can be evaluated by measuring mass loss, decrease in molecular weight, retention of mechanical properties, and/or tissue reaction. More critical for scaffold performance arc hydrolytic stability, thermal transitions crystallinity and orientation. Other determinants negatively affecting scaffold performance include, but not exclusively, monomeric impurities, cyclic and acyclic oligomers, structural defects and aging.

The medical device fashioned from the polymer compositions above may be significantly amorphous post extrusion or molding. Such devices may be subjected to controlled re-crystallization to induce incremental amounts of crystallinity and mechanical strength enhancement. Further crystallization can be induced by strain introduction at the time of device deployment. Such incremental re-crystallization may be employed either on a device “blank” prior to secondary or final fabrication (such as by laser cutting) or post such secondary fabrication. Crystallization (and thus mechanical properties) can also be maximized by strain induction such as by “cold” drawing polymeric tubing, hollow fiber, sheet or film, or monofilament prior to further fabrication. Crystallinity has been observed to contribute a greater stiffness in the medical device. Therefore, the polymer composition and steric complex of the scaffold has both amorphous and paracrystalline moieties. The initially semicrystalline polymer portion can be manipulated by the action of stretching or expansion of a given device. Yet an adequate amount of amorphous polymeric character is desirable for flexibility and elasticity of the polymeric device. The usual monomer components include lactide, glycolide, caprolactone, dioxanone, and trimethylene carbonate. The stent may also be constructed to allow relatively uniform exposure to local tissue or circulatory bioactive factors and enzymes perfusing and acting on the polymer structure during bioabsorption.

Advantageously, the rate of in situ breakdown kinetics of the polymeric matrix of an organ space implant, such as a cardiovascular stent, is sufficiently gradual to avoid tissue overload, inflammatory reactions or other more adverse consequences. In an embodiment, the scaffold is fabricated to survive at least one month.

The pharmaceutical compositions may be incorporated within the polymers by, for example, grafting to the polymer active sites, or coating. An embodiment of the polymer according to the invention affords attachment or incorporation the biological healing factors or other drugs in the polymeric matrix or a polymer coating.

In another embodiment, the composition may be constructed to structurally enclose or attach to drugs in the polymeric matrix. The purpose of such additives may to provide, for example with respect to a stent, treatment of the cardiovascular system or in vascular site in contact with the medical device polymer. The kind of enclosure or attachment of drugs in the polymer may determine the rate of release form the device. For example, the drug or other additive may be bound in the polymer matrix by various known methods including but not limited to covalent bonds, non-polar bonds as well as an ester or similar bioreversible bonding means.

In one embodiment, a bioabsorbable implantable medical device may be covered with a biodegradable and bioabsorbable coating containing one or more barrier layers where the polymer matrix contains one or more of the aforementioned pharmaceutical substances. In this embodiment, the barrier layer may comprise a suitable biodegradable material, including but not limited to, suitable biodegradable polymers including: polyesters such as PLA, PGA, PLGA, PPF, PCL, PCC, TMC and any copolymer of these; polycarboxylic acid, polyanhydrides including maleic anhydride polymers; polyorthoesters; poly-amino acids; polyethylene oxide; polyphosphacenes; polylactic acid, polyglycolic acid and copolymers and mixtures thereof such as poly(L-lactic acid) (PLLA), poly(D,L-lactide), poly(lactic acid-co-glycolic acid), 50/50 (DL-lactide-co-glycolide); polydixanone; polypropylene fumarate; polydepsipeptides; polycaprolactone and co-polymers and mixtures thereof such as poly(D,L-lactide-co-caprolactone) and polycaprolactone co-butylacrylate; polyhydroxybutyrate valerate and blends; polycarbonates such as tyrosine-derived polycarbonates and arylates, polyiminocarbonates, and polydimethyltrimethyl-carbonates; cyanoacrylate; calcium phosphates; polyglycosaminoglycans; macromolecules such as polysaccharides (including hyaluronic acid; cellulose, and hydroxypropylmethyl cellulose; gelatin; starches; dextrans; alginates and derivatives thereof), proteins and polypeptides; and mixtures and copolymers of any of the foregoing. The biodegradable polymer may also be a surface erodable polymer such as polyhydroxybutyrate and its copolymers, polycaprolactone, polyanhydrides (both crystalline and amorphous), maleic anhydride copolymers, and zinc-calcium phosphate. The number of barrier layers that the polymeric scaffold on a device may have depends on the amount of therapeutic need as dictated by the therapy required by the patient. For example, the longer the treatment, the more therapeutic substance required over a period of time, the more barrier layers to provide the pharmaceutical substance in a timely manner.

In another embodiment, the additive in the polymer composition may be in the form of a multiple component pharmaceutical composition within the matrix such as containing a last release pharmaceutical agent to retard early neointimal hyperplasia/smooth muscle cell migration and proliferation, and a secondary biostable matrix that releases a long acting agent for maintaining vessel patency or a positive blood vessel remodeling agent, such as endothelial nitric oxide synthase (eNOS), nitric oxide donors and derivatives such as aspirin or derivatives thereof, nitric oxide producing hydrogels, PPAR agonist such as PPAR-α gands, tissue plasminogen activator, statins such as atorvastatin, erythropoietin, darbepotin, serine proteinase-1 (SERP-1) and pravastatin, steroids, and/or antibiotics.

Pharmaceutical compositions may be incorporated into the polymers or may be coated on the surface of the polymers after mixing and extrusion by spraying, dipping or painting or microencapsulated and then blended into the polymer mixture. U.S. Pat. No. 6,020,385. If the pharmaceutical compositions are covalently bound to the polymer blend, they may be linked by hetero- or homo-bifunctional cross linking agents (see, http://www.piercenet.com/products/browse.cfm?fldID=020306).

Pharmaceutical agents that may be incorporated into the stents or may be coated on the stent include, but are not limited to, (i) pharmacological agents such as, (a) anti-thrombotic agents such as heparin, heparin derivatives, urokinase, and PPack (dextrophenylalanine proline arginine chloromethylketone); (b) anti-inflammatory agents such as dexamethasone, prednisolone, corticosterone, budesonide, estrogen, sulfasalazine and mesalamine; (c) antineoplastic/antiproliferative/anti-miotic agents such as paclitaxel, 5-fluorouracil, cisplatin, vinblastine, vincristine, epothilones, endostatin, angiostatin, angiopeptin, monoclonal antibodies capable of blocking smooth muscle cell proliferation, thymidine kinase inhibitors, rapamycin, 40-0-(2-Hydroxyethyl)rapamycin (everolimus), 40-0-Benzyl-rapamycin, 40-0(4′-Hydroxymethyl)benzyl-rapamycin, 40-0-[4′-(1,2-Dihydroxyethyl)]benzyl-rapamycin, 40-Allyl-rapamycin, 40-0-[3′-(2,2-Dimethyl-1,3-dioxolan-4(S)-yl-prop-2′-en-1′-yl]-20 rapamycin, (2′:E,4′S)-40-0-(4′,5′:Dihydroxypent-2′-en-1′-yl),rapamycin 40-0(2Hydroxy)ethoxycarbonylmethyl-rapamycin, 40-0-(3-Hydroxypropyl-rapamycin 40-0-((Hydroxy)hexyl-rapamycin 40-0-[2-(2-Hydroxy)ethoxy]ethyl-rapamycin, 40-0-[(3S)-2,2Dimethyldioxolan-3-yl]methyl-rapamycin, 40-0-[(2S)-2,3-Dihydroxyprop-1-yl]-rapamycin, 40-0-(2-Acctoxy)ethyl-rapamycin, 40-0-(2-Nicotinoyloxy)ethyl-rapamycin, 40-0-[2-(N-25 Morpholino)acetoxyethyl-rapamycin, 40-0-(2-N-Imidazolylacetoxy)ethyl-rapamycin, 40-0[2-(N-Methyl-N′-piperazinyl)acetoxy]ethyl-rapamycin, 39-0-Desmethyl-3.9,40-0,0 ethylene-rapamycin, (26R)-26-Dihydro-40-0-(2-hydroxy)ethyl-rapamycin, 28-O Methyrapamycin, 40-0-(2-Aminoethyl)-rapamycin, 40-0-(2-Acetaminoethyl)-rapamycin 40-0(2-Nicotinamidoethyl)-rapamycin, 40-0-(2-(N-Methyl-imidazo-2′ ylcarbethoxamido)ethyl)-30 rapamycin, 40-0-(2-Ethoxycarbonylaminoethyl)-rapamycin, 40-0-(2-Tolylsulfonamidoethyl)-rapamycin, 40-0-[2-(4′,5′-Dicarboethoxy-1′,2′;3′-triazol-1′-yl)-ethyl]rapamycin, 42-Epi-(telrazolyl)rapamycin (tacrolimus), and 42-[3-hydroxy-2-(hydroxymethyl)-2-methylpropanoate]rapamycin (temsirolimus) (WO2008/086369); (d) anesthetic agents such as lidocaine, bupivacaine and ropivacaine; (e) anti-coagulants such as D-Phe-Pro-Arg chloromethyl ketone, an RGD peptide-containing compound, heparin, hirudin, antithrombin compounds, platelet receptor antagonists, anti-thrombin antibodies, anti-platelet receptor antibodies, aspirin, prostaglandin inhibitors, platelet inhibitors and tick antiplatelet peptides; (f) vascular cell growth promoters such as growth factors, transcriptional activators, and translational promotors; (g) vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; (h) protein kinase and tyrosine kinase inhibitors (e.g., tyrphostins, genistein, quinoxalines); (i) prostacyclin analogs; (j) cholesterol-lowering agents; (k) angiopoietins; (l) antimicrobial agents such as triclosan, cephalosporins, aminoglycosides and nitrofurantoin; (m) cytotoxic agents, cytostatic agents and cell proliferation affectors; (n) vasodilating agents; and, (o) agents that interfere with endogenous vasoactive mechanisms, (ii) genetic therapeutic agents include anti-sense DNA and RNA as well as DNA coding for (a) anti-sense RNA, (b) tRNA or rRNA to replace defective or deficient endogenous molecules, (c) angiogenic factors including growth factors such as acidic and basic fibroblast growth factors, vascular endothelial growth factor, epidermal growth factor, transforming growth factor a and P, platelet-derived endothelial growth factor, platelet-derived growth factor, tumor necrosis factor a, hepatocyte growth factor and insulin-like growth factor, (d) cell cycle inhibitors including CD inhibitors, and (e) thymidine kinase (“TK”) and other agents useful for interfering with cell proliferation.

Other pharmaceutical agents that may be incorporated into the stents include, but are not limited to, acarbose, antigens, beta-receptor blockers, non-steroidal antiinflammatory drugs (NSAID, cardiac glycosides, acetylsalicylic acid, virustatics, aclarubicin, acyclovir, cisplatin, actinomycin, alpha- and beta-sympatomimetics, (dmeprazole, allopurinol, alprostadil, prostaglandins, amantadine, ambroxol, amlodipine, methotrexate, S-aminosalicylic acid, amitriptyline, amoxicillin, anastrozole, atenolol, azathioprine, balsalazide, beclomethasone, betahistine, bezafibrate, bicalutamide, diazepam and diazepam derivatives, budesonide, bufexamac, buprenorphine, methadone, calcium salts, potassium salts, magnesium salts, candesartan, carbamazepine, captopril, cefalosporins, cetirizine, chenodeoxycholic acid, ursodeoxycholic acid, theophylline and theophylline derivatives, trypsins, cimetidine, clarithromycin, clavulanic acid, clindamycin, clobutinol, clonidinc, cotrimoxazole, codeine, caffeine, vitamin D and derivatives of vitamin D, colestyramine, cromoglicic acid, coumarin and coumarin derivatives, cysteine, cytarabine, cyclophosphamide, cyclosporin, cyproterone, cytabarine, dapiprazole, desogestrel, desonide, dihydralazine, diltiazem, ergot alkaloids, dimenhydrinate, dimethyl sulphoxide, dimeticone, domperidone and domperidan derivatives, dopamine, doxazosin, doxorubizin, doxylamine, dapiprazole, benzodiazepines, diclofenac, glycoside antibiotics, desipramine, econazole, ACE inhibitors, enalapril, ephedrine, epinephrine, erythropoietin and erythropoietin derivatives, morphinans, calcium antagonists, irinotecan, modafmil, orlistat, peptide antibiotics, phenytoin, riluzoles, risedronate, sildenafil, topiramatc, macrolide antibiotics, oestrogen and oestrogen derivatives, progestogen and progestogen derivatives, testosterone and testosterone derivatives, androgen and androgen derivatives, ethenzamide, etofenamate, ctofibrate, fenofibrate, etofylHne, etoposide, famciclovir, famotidine, felodipine, fenofibrate, fentanyl, fenticonazole, gyrase inhibitors, fluconazole, fludarabine, fluarizine, fluorouracil, fluoxetine, flurbiprofen, ibuprofen, flutamide, fluvastatin, follitropin, formoterol, fosfomicin, furosemide, fusidic acid, gallopamil, ganciclovir, gemfibrozil, gentamicin, ginkgo, Saint John's wort, glibenclamide, urea derivatives as oral antidiabetics, glucagon, glucosamine and glucosamine derivatives, glutathione, glycerol and glycerol derivatives, hypothalamus hormones, goserelin, gyrase inhibitors, guanethidine, halofantrine, haloperidol, heparin and heparin derivatives, hyaluronic acid, hydralazine, hydrochlorothiazide and hydrochlorothiazide derivatives, salicylates, hydroxyzine, idarubicin, ifosfamide, imipramine, indometacin, indoramine, insulin, interferons, iodine and iodine derivatives, isoconazole, isoprenaline, glucitol and glucitol derivatives, itraconazole, ketoconazole, ketoprofen, ketotifen, lacidipine, lansoprazole, levodopa, levomethadone, thyroid hormones, lipoic acid and lipoic acid derivatives, lisinopril, lisuride, lofepramine, lomustine, loperamide, loratadine, maprotiline, mebendazole, mebeverine, meclozine, mefenamic acid, mefloquine, meloxicam, mcpindolol, meprobamate, meropenem, mesalazine, mesuximide, metamizole, metformin, methotrexate, methylphenidate, methylprednisolone, metixene, metoclopramide, metoprolol, metronidazole, mianserin, miconazole, minocycline, minoxidil, misoprostol, mitomycin, mizolastinc, moexipril, morphine and morphine derivatives, evening primrose, nalbuphine, naloxone, tilidine, naproxen, narcotine, natamycin, neostigmine, nicergoline, nicethamide, nifedipine, niflumic acid, nimodipine, nimorazole, nimustine, nisoldipine, adrenaline and adrenaline derivatives, norfloxacin, novamine sulfone, noscapine, nystatin, ofloxacin, olanzapine, olsalazine, omeprazole, omoconazole, ondansetron, oxaceprol, oxacillin, oxiconazole, oxymetazoline, pantoprazole, paracetamol, paroxetine, penciclovir, oral penicillins, pentazocine, pentifylline, pentoxifylline, perphenazine, pethidine, plant extracts, phenazone, pheniramine, barbituric acid derivatives, phenylbutazone, phenytoin, pimozide, pindolol, piperazine, piracetam, pirenzepine, piribedil, piroxicam, pramipexole, pravastatin, prazosin, procaine, promazine, propiverine, propranolol, propyphenazone, prostaglandins, protionamide, proxyphylline, quetiapine, quinapril, quinaprilat, ramipril, ranitidine, reproterol, reserpine, ribavirin, rifampicin, risperidone, ritonavir, ropinirole, roxatidine, roxithromycin, ruscogenin, rutoside and rutoside derivatives, sabadilla, salbutamol, salmeterol, scopolamine, selegiline, sertaconazole, sertindole, sertralion, silicates, sildenafil, simvastatin, sitosterol, sotalol, spaglumic acid, sparfloxacin, spectinomycin, spiramycin, spirapril, spironolactone, stavudine, streptomycin, sucralfate, sufentanil, sulbactam, sulphonamides, sulfasalazine, sulpiride, sultamicillin, sultiam, sumatriptan, suxamethonium chloride, tacrine, tacrolimus, taliolol, tamoxifen, taurolidine, tazarotene, temazepam, teniposide, tenoxicam, terazosin, terbinafine, terbutaline, terfenadine, terlipressin, tertatolol, tctracyclins, teryzoline, theobromine, theophylline, butizine, thiamazole, phenothiazines, thiotepa, tiagabine, tiapride, propionic acid derivatives, ticlopidine, timolol, tinidazole, tioconazole, tioguanine, tioxolone, tiropramide, tizanidine, tolazoline, tolbutamide, tolcapone, tolnaftate, tolperisone, topotecan, torasemide, antioestrogens, tramadol, tramazoline, trandolapril, tranylcypromine, trapidil, trazodone, triamcinolone and triamcinolone derivatives, triamterene, trifluperidol, trifluridine, trimethoprim, trimipramine, tripelennamine, triprolidine, trifosfamide, tromantadine, trometamol, tropalpin, troxerutine, tulobutcrol, tyramine, tyrothricin, urapidil, ursodeoxycholic acid, chenodeoxycholic acid, valaciclovir, valproic acid, vancomycin, vecuronium chloride, Viagra, venlafaxine, vcrapamil, vidarabine, vigabatrin, viloazinc, vinblastine, vincaminc, vincristine, vindesine, vinorclbinc, vinpocetine, viquidil, warfarin, xantinol nicotinate, xipamide, zafirlukast, zalcitabine, zidovudine, zolmitriptan, Zolpidem, zoplicone, zotipine and the like. See, e.g., U.S. Pat. Nos. 6,897,205, 6,838,528 and 6,497,729.

The stent may also be coated with at least one type of antibodies. For example, the stent may be coated with antibodies or polymeric matrices which are capable of capturing circulating endothelial cells. U.S. Pat. No. 7,037,772 (see also, U.S. Patent Publications Nos. 20070213801, 200701196422, 20070191932, 20070156232, 20070141107, 20070055367, 20070042017, 20060135476, 20060121012).

The stent of the present invention may also be formed from metal such as nickel-titanium (Ni—Ti). A metal composition and process of manufacturing the device is disclosed in U.S. Pat. No. 6,013,854. The super elastic metal for the device is preferably a super elastic alloy. A super elastic alloy is generally called “a shape-memory alloy” and resumes its original shape after being deformed to such a degree that an ordinary metal undergoes permanent deformation. Super elastic alloys useful in the invention include: Elgiloy® and Phynox® spring alloys (Elgiloy® alloy is available from Carpenter Technology Corporation of Reading Pa.; Phynox® alloy is available from Metal Imphy of Imphy, France), 316 stainless steel and MP35N alloy which are available from Carpenter Technology corporation and Latrobe Steel Company of Latrobe, Pa., and superelastic Nitinol nickel-titanium alloy which is available from Shape Memory Applications of Santa Clara, Calif. U.S. Pat. No. 5,891,191.

The device of the present invention may be manufactured in numerous ways. The device may be formed from a tube by removing various portions of the tube's wall to form the patterns described herein. The resulting device will thus be formed from a single contiguous piece of material, eliminating the need for connecting various segments together. Material from the tube wall may be removed using various techniques including laser (YAG laser for example), electrical discharge, chemical etching, metal cutting, a combination of these techniques, or other well known techniques. U.S. Pat. Nos. 5,879,381 and 6,117,165 which are hereby incorporated in their entirety by reference. Forming stents in this manner allows for creation of a substantially stress-free structure. In particular, the length may be adapted to that of the diseased part of the lumen in which the stent is to be placed. This may avoid using separate stents to cover the total diseased area.

In an alternate embodiment, a method for fabricating a tube-shaped stent comprising: preparing a racemic poly-lactide mixture; fabricating a biodegradable polymer tube of the racemic poly-lactide mixture; laser cutting the tube until such scaffold is formed. In this embodiment, the fabrication of the scaffold can be performed using a molding technique, which is substantially solvent-free, or an extrusion technique.

Reference is also made, and thereby incorporated in their entirety into this application, to U.S. Pat. Nos. 7,329,277, 7,169,175, 7,846,197, 7,846,361, 7,833,260, 6,0254,688, 6,254,631, 6,132,461, 6,821,292, 6,245,103 and 7,279,005. In addition, U.S. patent application Ser. Nos. 11/781,230, 12/507,663, 12/508,442, 12/986,862, 11/781,233, 12/434,596, 11/875,887, 12/464,042, 12/576,965, 12/578,432, 11/875,892, 11/781,229, 11/781,353, 11/781,232, 11/781,234, 12/603,279, 12/779,767 and 11/454,968, as well as U.S. Patent Publication No. 2001/0029397, are also incorporated in their entirety.

The scope of the present invention is not limited by what has been specifically shown and described hereinabove. Those skilled in the art will recognize that there are suitable alternatives to the depicted examples of materials, configurations, constructions and dimensions. Numerous references, including patents and various publications, are cited and discussed in the description of this invention. The citation and discussion of such references is provided merely to clarify the description of the present invention and is not an admission that any reference is prior art to the invention described herein. All references cited and discussed in this specification are incorporated herein by reference in their entirety. Variations, modifications and other implementations of what is described herein will occur to those of ordinary skill in the art without departing from the spirit and scope of the invention. While certain embodiments of the present invention have been shown and described, it will be obvious to those skilled in the art that changes and modifications may be made without departing from the spirit and scope of the invention. The matter set forth in the foregoing description and accompanying drawings is offered by way of illustration only and not as a limitation.

What is claimed is:

1. A stent comprising at least one bioabsorbable polymer, and comprising: a plurality of first circumferential elements and a plurality of second circumferential elements, the first circumferential elements and the second circumferential elements being arranged in an alternating pattern, adjacent first and second circumferential elements being connected by at least one connection element, the circumference of the first circumferential element being greater than the circumference of the second circumferential element, wherein the first circumferential element comprises a plurality of undulations, and wherein the second circumferential element comprises a hoop-like structure when expanded.

2. The stent of claim 1, wherein at least one undulation in the first circumferential element comprises at least two segments forming at least one angle.

3. The stent of claim 2, wherein the angle formed by the segments ranges from about 30° to about 180° when the stent is unexpanded.

4. The stem of claim 3, wherein the angle formed by the segments ranges from about 60° to about 150° when the stent is unexpanded.

5. The stem of claim 2, wherein the segments are equal or unequal in length.

6. The stent of claim 2, wherein the segment is linear or curvilinear.

7. The stem of claim 1, wherein the first circumferential element comprises a plurality of first undulations and a plurality of second undulations.

8. The stent of claim 1, wherein the first circumferential element comprises a plurality of first undulations, a plurality of second undulations and a plurality of third undulations.

9. The stent of claim 1, wherein adjacent first and second circumferential elements are connected by two connection elements.

10. The stent of claim 1, wherein adjacent first and second circumferential elements are connected by three connection elements.

11. The stent of claim 1, wherein adjacent first circumferential element and second circumferential element are connected by at least one first strut.

12. The stent of claim 1, wherein adjacent first circumferential element and second circumferential element are directly connected.

13. The stent of claim 1, wherein the second circumferential element is in a sinusoidal pattern when unexpanded.

14. The stent of claim 1, wherein the second circumferential element comprises at least one indentation.

15. The stent of claim 14, wherein the indentation is a notched structure.

16. The stent of claim 1, further comprising at least one end zone positioned at either end or both ends of the stent, wherein the end zone comprises a plurality of cylindrical elements.

17. The stent of claim 16, wherein the cylindrical element is in a sinusoidal pattern when unexpanded.

18. The stent of claim 16, wherein the end zone is attached to the first circumferential element by at least one second strut.

19. The stem of claim 16, wherein the end zone is directly attached to the first circumferential element.

20. The stent of claim 16, wherein the end zone further comprises at least one radiopaque marker.

21. The stent of claim 1, wherein the stent is crimped.

22. The stent of claim 1, wherein the stent is expanded.

23. A stent comprising at least one bioabsorbable polymer, and comprising: a plurality of first circumferential elements, a plurality of second circumferential elements and a plurality of third circumferential elements, the first, second and third circumferential elements forming a group which is repeated at least three times, adjacent first and second circumferential elements being connected by at least one connection element, adjacent first and third circumferential elements being connected by at least one connection element, adjacent second and third circumferential elements being connected by at least one connection element, the circumference of the first circumferential element being greater than the circumference of the second and third circumferential elements, wherein the first circumferential element comprises a plurality of undulations, wherein the second circumferential element comprises a hoop-like structure when expanded, and wherein the third circumferential element comprises a hoop-like structure when expanded.

24. The stent of claim 23, wherein at least one undulation in the first circumferential element comprises at least two segments forming at least one angle.

25. The stent of claim 23, wherein the angle formed by the segments ranges from about 30° to about 180° when the stent is crimped.

26. The stem of claim 23, wherein the angle formed by the segments ranges from about 60° to about 150° when the stem is crimped.

27. The stent of claim 24, wherein the segments are equal or unequal in length.

28. The stem of claim 24, wherein the segment is linear or curvilinear.

29. The stent of claim 24, wherein the first circumferential elements comprise a plurality of first undulations and a plurality of second undulations.

30. The stent of claim 24, wherein the first circumferential elements comprise a plurality of first undulations, a plurality of second undulations and a plurality of third undulations.

31. The stein of claim 24, wherein adjacent first and second circumferential elements are connected by two connection elements.

32. The stent of claim 24, wherein adjacent first and third circumferential elements are connected by two connection elements.

33. The stent of claim 24, wherein adjacent second and third circumferential elements are connected by six connection elements.

34. The stent of claim 24, wherein adjacent second circumferential element and third circumferential element are connected by at least one first strut.

35. The stent of claim 24, wherein adjacent first circumferential element and second circumferential element are directly connected.

36. The stent of claim 24, wherein adjacent first circumferential element and third circumferential element are directly connected.

37. The stent of claim 24, wherein the second circumferential element is in a sinusoidal pattern when unexpanded.

38. The stent of claim 24, wherein the third circumferential element is in a sinusoidal pattern when unexpanded.

39. The stent of claim 24, further comprising at least one end zone positioned at either end or both ends of the stent, wherein the end zone comprises a plurality of cylindrical elements.

40. The stent of claim 39, wherein the cylindrical element is in a sinusoidal pattern when unexpanded.

41. The stent of claim 39, wherein the end zone is directly connected to the first circumferential element.

42. The stent of claim 39, wherein the end zone further comprises at least one radiopaque marker.

43. The stent of claim 23, wherein the stent is crimped.

44. The stent of claim 23, wherein the stent is expanded.
